SEQUENCE LISTING (36 sequences)

SEQ ID NO: 1 - DNA, 582 bp, SARS-CoV-2
aatatcacaaacctgtgcccctttggcgaggtgttcaacgccacccgcttcgccagcgtgtatgcctggaatcggaagagaatctccaactgcgtggccgattattctgtgctgtacaactccgcctctttcagcacctttaagtgctatggcgtgtcccccacaaagctgaatgacctgtgctttaccaacgtgtacgccgattctttcgtgatccggggcgacgaggtgagacagatcgcccctggccagacaggcaagatcgccgattacaattataagctgccagacgatttcaccggctgcgtgatcgcctggaacagcaacaatctggactccaaagtgggcggcaactacaattatctgtacaggctgtttcgcaagtctaatctgaagcccttcgagcgggacatctctacagaaatctaccaggccggcagcaccccttgcaatggcgtggagggctttaactgttatttccccctgcagagctacggctttcagcctaccaacggcgtgggctatcagccatacagagtggtggtgctgagcttcgagctgctgcacgcaccagcaacagtg

SEQ ID NO: 2 - protein, 194 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 3 - DNA, 1941 bp, SARS-CoV-2
cagtgcgtgaatctgaccacaaggacccagctgccacctgcatacaccaactcctttacaaggggcgtgtactatccagataaggtgttccgcagctccgtgctgcacagcacacaggacctgtttctgcccttcttttccaatgtgacctggttccacgccatccacgtgtctggcaccaacggcacaaagcggtttgataatccagtgctgccctttaacgacggcgtgtacttcgccagcaccgagaagtccaatatcatcagaggctggatcttcggcaccacactggattctaagacacagagcctgctgatcgtgaacaatgccaccaacgtggtcatcaaggtgtgcgagttccagttttgtaacgacccatttctgggcgtgtactatcacaagaacaataagtcctggatggagtctgagttcagagtgtattctagcgccaacaattgtacatttgagtacgtgagccagcccttcctgatggatctggagggcaagcagggcaacttcaagaacctgagggagttcgtgtttaagaatatcgacggctacttcaaaatctactctaagcacacccctatcaacctggtgcgcgatctgccacagggctttagcgccctggagcctctggtggacctgccaatcggcatcaacatcaccaggttccagacactgctggccctgcaccgctcctacctgacacctggcgattcctctagcggatggaccgccggcgctgccgcctactatgtgggctatctgcagccaaggacctttctgctgaagtacaacgagaatggcaccatcacagacgcagtggattgcgccctggaccccctgagcgagaccaagtgtacactgaagtccttcaccgtggagaagggcatctaccagacatccaattttcgggtgcagcctaccgagtctatcgtgagattcccaaatatcacaaacctgtgcccctttggcgaggtgttcaacgccacccgcttcgccagcgtgtatgcctggaatcggaagagaatctccaactgcgtggccgattattctgtgctgtacaactccgcctctttcagcacctttaagtgctatggcgtgtcccccacaaagctgaatgacctgtgctttaccaacgtgtacgccgattctttcgtgatccggggcgacgaggtgagacagatcgcccctggccagacaggcaagatcgccgattacaattataagctgccagacgatttcaccggctgcgtgatcgcctggaacagcaacaatctggactccaaagtgggcggcaactacaattatctgtacaggctgtttcgcaagtctaatctgaagcccttcgagcgggacatctctacagaaatctaccaggccggcagcaccccttgcaatggcgtggagggctttaactgttatttccccctgcagagctacggctttcagcctaccaacggcgtgggctatcagccatacagagtggtggtgctgagcttcgagctgctgcacgcaccagcaacagtgtgcggacctaagaagtccaccaatctggtgaagaacaagtgcgtgaacttcaacttcaacggactgaccggcacaggcgtgctgaccgagagcaacaagaagttcctgccttttcagcagttcggccgggacatcgccgataccacagacgccgtgagagatccccagaccctggagatcctggacatcacaccttgctcttttggcggcgtgagcgtgatcacacccggcaccaatacatccaaccaggtggccgtgctgtatcaggatgtgaattgtaccgaggtgccagtggcaatccacgcagaccagctgaccccaacatggagggtgtactccaccggctctaacgtgttccagacacgcgccggatgtctgatcggagcagagcacgtgaacaattcctat

SEQ ID NO: 4 - protein, 647 aa, SARS-CoV-2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSY

SEQ ID NO: 5 - protein, 23 aa, Homo sapiens
MDAMKRGLCCVLLLCGAVFVSAS

SEQ ID NO: 6 - protein, 20 aa, SARS-CoV-2
NITNLCPFGEVFNATRFASV

SEQ ID NO: 7 - protein, 20 aa, SARS-CoV-2
VFNATRFASVYAWNRKRISN

SEQ ID NO: 8 - protein, 20 aa, SARS-CoV-2
YAWNRKRISNCVADYSVLYN

SEQ ID NO: 9 - protein, 20 aa, SARS-CoV-2
CVADYSVLYNSASFSTFKCY

SEQ ID NO: 10 - protein, 20 aa, SARS-CoV-2
SASFSTFKCYGVSPTKLNDL

SEQ ID NO: 11 - protein, 20 aa, SARS-CoV-2
GVSPTKLNDLCFTNVYADSF

SEQ ID NO: 12 - protein, 20 aa, SARS-CoV-2
CFTNVYADSFVIRGDEVRQI

SEQ ID NO: 13 - protein, 20 aa, SARS-CoV-2
VIRGDEVRQIAPGQTGKIAD

SEQ ID NO: 14 - protein, 20 aa, SARS-CoV-2
APGQTGKIADYNYKLPDDFT

SEQ ID NO: 15 - protein, 20 aa, SARS-CoV-2
YNYKLPDDFTGCVIAWNSNN

SEQ ID NO: 16 - protein, 20 aa, SARS-CoV-2
GCVIAWNSNNLDSKVGGNYN

SEQ ID NO: 17 - protein, 20 aa, SARS-CoV-2
LDSKVGGNYNYLYRLFRKSN

SEQ ID NO: 18 - protein, 20 aa, SARS-CoV-2
YLYRLFRKSNLKPFERDIST

SEQ ID NO: 19 - protein, 20 aa, SARS-CoV-2
LKPFERDISTEIYQAGSTPC

SEQ ID NO: 20 - protein, 20 aa, SARS-CoV-2
EIYQAGSTPCNGVEGFNCYF

SEQ ID NO: 21 - protein, 20 aa, SARS-CoV-2
NGVEGFNCYFPLQSYGFQPT

SEQ ID NO: 22 - protein, 20 aa, SARS-CoV-2
PLQSYGFQPTNGVGYQPYRV

SEQ ID NO: 23 - protein, 20 aa, SARS-CoV-2
YQPYRVVVLSFELLHAPATV

SEQ ID NO: 24 - protein, 27 aa, Artificial Sequence
  C terminal domain of T4 fibritin
GYIPEAPRDGQAYVRKDGEWVLLSTFL

SEQ ID NO: 25 - protein, 29 aa, Artificial Sequence
  C-terminal domain of T4 fibritin
GSGYIPEAPRDGQAYVRKDGEWVLLSTFL

SEQ ID NO: 26 - protein, 32 aa, Artificial Sequence
  GCN4
MKQIEDKIEEILSKIYHIENEIARIKKLIGEV

SEQ ID NO: 27 - protein, 28 aa, Artificial Sequence
  IQ domain
RMKQIEDKIEEIESKQKKIENEIARIKK

SEQ ID NO: 28 - protein, 24 aa, Artificial Sequence
  IZ domain
IKKEIEAIKKEQEAIKKKIEAIEK

SEQ ID NO: 29 - protein, 229 aa, Homo sapiens
RSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 234 aa, Mus musculus
RSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 225 aa, Oryctolagus cuniculus
RSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 32 - protein, 302 aa, Homo sapiens
HLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 33 - protein, 104 aa, Vibrio cholerae
MTPQNITDLCAEYHNTQIHTLNDKIFSYTESLAGKREMAIITFKNGATFQVEVPGSQHIDSQKKAIERMKDTLRIAYLTEAKVEKLCVWNNKTPRAIAAISMAN

SEQ ID NO: 34 - protein, 5 aa, Artificial Sequence
  Flexible linker (GGGGS)n, wherein n is an integer between 0 and 8
GGGGS

SEQ ID NO: 35 - protein, 193 aa, SARS-CoV
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 36 - protein, 212 aa, MERS-CoV
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

(19) United States
(12) Patent Application Publication
(10) Pub. No.: US 2023/0346916 A1
(43) Pub. Date: Nov. 2, 2023

(54) IMMUNOGENIC COMPOSITIONS AGAINST SEVERE ACUTE RESPIRATORY SYNDROME CORONAVIRUS 2

(71) Applicant: New York Blood Center, Inc., New York, NY (US)

(72) Inventors: Lanying DU, New York, NY (US); Wanbo TAI, New York, NY (US); Christopher HILLYER, New York, NY (US); Larry LUCHSINGER, New York, NY (US); Shibo JIANG, New York, NY (US)

(21) Appl. No.: 17/908,455

(22) PCT Filed: Mar. 4, 2021
(86) PCT No.: PCT/US2021/020806

§ 371 Date: Aug. 31, 2022

Related U.S. Application Data
(60) Provisional application No. 62/985,758, filed on Mar. 5, 2020.
(60) Provisional application No. 63/021,412, filed on May 7, 2020.
(60) Provisional application No. 63/055,742, filed on Jul. 23, 2020.

(51) Int. Cl. A61K 39/215; C07K 14/005; A61P 31/14

(52) U.S. Cl. CPC A61K 39/215; C07K 14/005; A61P 31/14; C12N 2770/20022; C07K 2319/30; C12N 2770/20034; C12N 2770/20071; A61K 2039/545

(57) ABSTRACT

Disclosed herein are immunogenic compositions for the prevention of infection with SARS-CoV-2. The immunogenic compositions comprise polypeptides, RNA, or DNA and are capable of inducing a long-term and broad immune response in a subject.

### CROSS-REFERENCE TO RELATED APPLICATIONS

This application claims the benefit of U. S. Provisional Application Nos. 62/985,758, filed Mar. 5, 2020, 63/021,412, filed May 7, 2020, and 63/055,742, filed Jul. 23, 2020, the entire contents of which are each incorporated by reference herein.

### FEDERALLY SPONSORED RESEARCH OR DEVELOPMENT

This invention was made with government support under Grant Nos. R01A1137472 and R01A1139092 awarded by the National Institutes of Health. The government has certain rights in the invention.

### BACKGROUND

Three highly pathogenic human coronaviruses (CoVs) have been identified so far, including Middle East respiratory syndrome coronavirus (MERS-CoV), severe acute respiratory syndrome (SARS) coronavirus (SARS-CoV), and SARS-CoV-2 (also known as 2019 novel coronavirus (2019-nCoV), as previously termed by the World Health Organization (WHO)). Among them, SARS-CoV was first reported in Guangdong, China in 2002. SARS-CoV caused human-to-human transmission and resulted in the 2003 outbreak with about 10% case fatality rate (CFR), while MERS-CoV was reported in Saudi Arabia in June 2012. Though exhibiting limited human-to-human transmission, MERS-CoV showed a CFR of about 34.4%.

SARS-CoV recognizes angiotensin-converting enzyme 2 (ACE2) as its cellular receptor for host cell entry. The viral surface spike (S) protein plays a key role in viral infection and pathogenesis. The S protein has 51, including the receptor-binding domain (RBD), and S2 subunits. 51 binds ACE2 receptor via the RBD, and S2 mediates virus-cell membrane fusion. We previously demonstrated that S proteins, particularly RBDs, of SARS-CoV and MERS-CoV serve as an important target for the development of effective vaccines against SARS-CoV and MERS-Coy infection, respectively.

SARS-CoV-2, the causative agent of Coronavirus Disease 2019 (COVID-19), is spreading globally with rapid human-to-human transmission, leading to worldwide panic and severe economic loss. SARS-CoV-2 is believed to have originated from bats, using bats as its natural reservoir, but its intermediate host is still under investigation. Although several vaccines have been approved for use in humans to prevent SARS-CoV-2-caused COVID-19 disease, further safe, effective, and broad-spectrum vaccines are still urgently needed to prevent continuous threat of COVID-19 spread, particularly variant strains of SARS-CoV-2 infection.

### SUMMARY

SARS-CoV-2 shares about 79.6% sequence identity with SARS-CoV, and it also utilizes ACE2 as its cellular receptor. Here, we have characterized the SARS-CoV-2 S protein receptor-binding domain (RBD; residues 331-524) and demonstrated that it binds both human ACE2 (hACE2) and bat ACE2 (bACE2) receptors. The binding between SARS-CoV-2 RBD and ACE2 is much stronger than that between SARS-CoV RBD and ACE2. We have also designed two nucleoside-modified mRNAs respectively encoding SARS-CoV-2 RBD and S1. Our data show that a lipid nanoparticle (LNP)-encapsulated SARS-CoV-2 RBD-based mRNA vaccine (RBD mRNA-LNPs) was stable with broad-spectrum expression in different cells. It also induced potent T follicular helper (Tfh) and germinal center (GC) B cell responses and long-term and broad neutralizing antibodies against infections of the original prototype virus strain as well as recent variant virus strains of SARS-CoV-2. Moreover, nucleoside-modified RBD mRNA-LNPs elicited SARS-CoV-2 RBD-specific cellular immune responses.

The outbreak of Coronavirus Disease 2019 (COVID-19) has spread globally and has posed a serious threat to worldwide public health with severe economic consequences, calling for the development of safe and effective prophylactics, therapeutics, and vaccines against infection of its causative agent, severe acute respiratory syndrome coronavirus 2 (SARS-CoV-2), previously known as 2019 novel coronavirus (2019-nCoV). Here, we identified the RBD in SARS-CoV-2 S protein and found that the RBD protein bound strongly to human and bat angiotensin-converting enzyme 2 (ACE2) receptors. SARS-CoV-2 RBD protein exhibited significantly higher binding affinity to ACE2 receptor than SARS-CoV RBD and could block the binding and, hence, attachment of SARS-CoV-2 and SARS-CoV to ACE2-expressing cells, thus inhibiting their infection of host cells. SARS-CoV RBD-specific polyclonal and monoclonal antibodies (mAbs) cross-react with SARS-CoV-2 RBD protein, and SARS-CoV RBD-induced antisera and mAbs cross-neutralize SARS-CoV-2 infection, suggesting the potential to develop SARS-CoV RBD-based vaccines and antibodies for prevention and treatment of SARS-CoV-2 and SARS-CoV infection.

Messenger RNA (mRNA) has emerged as useful platform for rapid development of much needed vaccines with cost-effective manufacturing capacity to prevent viral pathogens. We made two nucleoside-modified mRNA vaccines encoding S1 and RBD in S1 of SARS-CoV-2, encapsulating them with lipid nanoparticles (LNPs) for delivery. Compared with a S1 mRNA-LNP vaccine, a RBD mRNA-LNP vaccine maintained strong stability and long-term expression, broadly expressed in a variety of human, monkey and bat cells. It elicited Tfh and GC B cell responses and potent and broad neutralizing antibodies in mice against infection of prototype and variant strains of SARS-CoV-2, as well as cross-neutralizing SARS-CoV infection. The nucleoside-modified RBD mRNA-LNPs induced SARS-CoV-2 RBD-specific cellular immune responses.

Some embodiments comprise a polypeptide comprising a SARS-CoV-2 RBD. In some embodiments, the polypeptide comprising a SARS-CoV-2 RBD is an immunogenic polypeptide, that is, it is an immunogen. In some embodiments, the polypeptide comprises a whole, or nearly whole, S1 segment of the SARS-CoV-2 spike protein, but not the whole spike protein (see FIG. 6A). In some embodiments, the whole S1 segment comprises amino acids 14-694 of the spike protein. In some embodiments, the nearly whole S1 segment comprises amino acids 14-660.

In some embodiments, the polypeptide comprises a SARS-CoV-2 RBD, but not a whole S1 segment of the spike protein. In some embodiments, the RBD comprises amino acids 331-524 of the SARS-CoV-2 spike protein. In other embodiments, the RBD comprises amino acids 336-516 or amino acids 333-526 of the SARS-CoV-2 spike protein.

In some embodiments, the polypeptide comprising a SARS-CoV-2 RBD comprises further functional portions. In some embodiments, the polypeptide comprises an immunopotentiator portion. In various aspects, the immunopotentiator portion can comprise a human, mouse, or rabbit IgG Fc region, a human C3d domain, or a cholera toxin b subunit. In some embodiments, the immunopotentiator portion can be referred to as means for immunopotentiation. Some embodiments specifically include one or more of these immunopotentiators. Other embodiments specifically exclude one or more of these immunopotentiators. In some embodiments, the RBD comprising polypeptide is fused directly to the further functional portions, while in other embodiments a linker is interposed between the RGB sequence and the immunopotentiator sequence. Is some embodiments, the interposed linker is (GGGGS)ₙ where n equals from 1 to 8. Some embodiments comprise a SARS-CoV-2 RBD polypeptide sequence fused to a human IgG Fc sequence.

In various embodiments, the polypeptides may be referred to as means for inducing an anti-RBD immune response, means for binding to human ACE2 (hACE2) receptor, or means for inhibiting binding to human ACE2 (hACE2) receptor. Some such embodiments specifically include one of more genera, sub-genera, or species of polypeptide comprising a SARS-CoV-2 RBD. Some such embodiments specifically exclude one of more genera, sub-genera, or species of polypeptide comprising a SARS-CoV-2 RBD.

Some embodiments comprise a nucleic acid sequence encoding a polypeptide comprising a SARS-CoV-2 RBD. In some embodiments the nucleic acid is RNA, for example, a mRNA. In some embodiments, the nucleic acid is DNA. In one aspect of the DNA embodiments, the DNA encodes an mRNA embodiment. In another aspect of the DNA embodiments, the DNA encodes an immunogenic polypeptide embodiment as described herein. In some embodiments, the encoded polypeptide is immunogenic, such that a polynucleotide from which the immunogenic polypeptide can be expressed is an immunogen.

In some mRNA embodiments, in addition to the SARS-CoV-2 RBD-encoding sequence, the polynucleotide comprises one or more of a 5′-cap, a 5′ untranslated region, a heterologous signal peptide, a 3′ untranslated region, and a 3′-polyA tail (see FIG. 6A). In one aspect, the heterologous signal peptide is the signal peptide from tissue plasminogen activator (tPA). In various embodiments, the SARS-CoV-2 RBD-encoding sequence of the mRNA encodes any of the above-mentioned SARS-CoV-2 RBD polypeptides, including the 51 segment, the RBD itself, having reference or variant sequence. In some embodiments, these SARS-CoV-2 RBD-encoding sequences are referred to as means for encoding a polypeptide comprising a SARS-CoV-2 RBD. Some such embodiments specifically include one of more genera, sub-genera, or species encoding a polypeptide comprising a SARS-CoV-2 RBD. Some such embodiments specifically exclude one of more genera, sub-genera, or species encoding a polypeptide comprising a SARS-CoV-2 RBD. In some embodiments, the mRNA comprising a SARS-CoV-2 RBD-encoding sequence are referred to as means for expressing a polypeptide comprising a SARS-CoV-2 RBD. Some such embodiments specifically include one of more genera, sub-genera, or species of mRNA comprising a SARS-CoV-2 RBD-encoding sequence. Some such embodiments specifically exclude one of more genera, sub-genera, or species of mRNA comprising a SARS-CoV-2 RBD-encoding sequence.

In some embodiments, the mRNA comprises a SARS-CoV-2 RBD-encoding sequence comprising pseudouridine nucleosides in place of the uridine nucleosides that would be found in a naturally-occurring polynucleotide.

It is to be understood that numbering of amino acid and nucleic acid sequences follows that of reference sequences as identified herein below, but that any particular isolate of virus may vary in the residue present at any particular position or in the number of residues between reference positions. Some embodiments encompass such variation. Other embodiments correspond to a SARS-CoV-2 RBD-comprising polypeptide sequence of a particular reference sequence or variant sequence. Some nucleic acids encoding a SARS-CoV-2 RBD-comprising polypeptide have been codon-optimized to facilitate expression.

Some embodiments are immunogenic compositions comprising an immunogen for a SARS-CoV-2 RBD. In some embodiments, the immunogen is an immunogenic polypeptide comprising a SARS-CoV-2 RBD. In some embodiments, the immunogen is a polynucleotide encoding, and capable of expressing, an immunogenic polypeptide comprising a SARS-CoV-2 RBD. In some embodiments, the polynucleotide immunogen is an mRNA. Typically, the immunogenic composition further comprises one or more of a pharmaceutically acceptable carriers, buffers, or excipients. In some embodiments, the immunogenic composition further comprises an adjuvant.

In some embodiments in which the immunogenic composition comprises an mRNA, the mRNA is encapsulated in a lipid nanoparticle (LNP).

Some embodiments are methods of making an immunogenic composition or vaccine. Some such embodiments comprise expressing a polypeptide comprising a SARS-CoV-2 RBD in an expression system, for example a mammalian cell expression system, an insect cell expression system, or a bacterial expression system. Such methods may further comprise purifying the polypeptide comprising a SARS-CoV-2 RBD. In some aspects, when the polypeptide comprising a SARS-CoV-2 RBD is a fusion protein comprising an IgG Fc, purification can comprise affinity purification with, for example, protein A or Protein G. Affinity chromatography directed to other fusion partners or tags, such as a GST protein or a His tag, can also be used. Anti-RBD antibodies can also be used for affinity reagents. Other liquid chromatography methods may also be applied.

Further embodiments of making an immunogenic composition or vaccine comprise synthesizing an mRNA encoding a SARS-CoV-2 RBD-comprising polypeptide. Some embodiments comprise linearizing a DNA plasmid encoding the mRNA operably linked to transcription control elements, such as a promoter and polyadenylation signal. In some embodiments, pseudouridine-5′-triphosphate is included in the transcription reaction instead of uridine-5′-triphosphate. In some embodiments, T7 polymerase is used for transcription. Some embodiments further comprise a capping reaction and/or a polyadenylation reaction. Some embodiments further comprise encapsulating the mRNA in an LNP. In some aspects, a mixture of lipids in an organic solvent, for example, ethanol, is mixed with the mRNA in an aqueous solution, for example, at a ratio of 1:3 organic phase:aqueous phase, to form the LNP-encapsulated mRNA.

In some embodiments, making an immunogenic composition or vaccine further comprises combining the polypeptide or mRNA with one or more pharmaceutically acceptable carriers, buffers, or excipients. In some embodiments, making an immunogenic composition or vaccine further comprises combining the polypeptide or mRNA with an adjuvant.

Some embodiments are methods of inducing an immune response recognizing the SARS-CoV-2 RBD as present in the SARS-CoV-2 virion in a subject, comprising administering one of the immunogens, or immunogenic compositions, disclosed herein to the subject. In some embodiments, the immune response comprises a SARS-CoV-2 RBD-specific humoral immune response. In various aspects, the humoral immune response comprises production of SARS-CoV-2 RBD-specific IgG, IgG1, and/or IgG2a antibody. In some embodiments, the immune response comprises a SARS-CoV-2 RBD-specific cellular immune response. In various aspects, the cellular immune response production of SARS-CoV-2 RBD-specific CD4⁺ and/or CD8⁺ T cell response. In further aspects, the CD4⁺ and/or CD8⁺ T cells are CD45⁺ T cells. In still further aspects, the CD45⁺, CD4⁺ and/or CD45⁺, CD8⁺ T cells produce interferon-γ (IFNγ), tumor necrosis factor α (TNFα), and/or interleukin 4 (IL-4). In some embodiments, the immune response is a neutralizing response, blocking or reducing infectivity of SARS-CoV-2 virus.

In some embodiments, the method is prophylactic, the immunogen or immunogenic composition being administered prior to infection, to prevent or reduce the severity or duration of SARS-CoV-2 infection. In some embodiments, the method is therapeutic, the immunogen or immunogenic composition being administered prior to infection, to reduce the severity or duration of SARS-CoV-2 infection. Although the aim of these methods is to address SARS-CoV-2 infection, due to cross-reactivity and cross neutralization of SARS-CoV, in further embodiments, these methods of inducing an immune response to recognizing the SARS-CoV-2 RBD can be similarly applied to address SARS-CoV infection.

Some embodiments of methods of inducing an immune response recognizing the SARS-CoV-2 RBD comprise a single administration of the immunogen or immunogenic composition. Other embodiments comprise multiple administrations; an initial priming dose and one or more subsequent boosting doses. In some embodiments, the priming dose and the boosting dose(s) comprise the same immunogen, while in other embodiments different immunogens are used for the prime and the boost, for example, mRNA for one and polypeptide for the other. Some embodiments comprise two administrations, a prime followed by a boost 3-4 weeks later. In some embodiments, the prime and the boost are administered by the same route of administration, for example intradermal, intramuscular, intravenous, intranasal, or subcutaneous administration. In other embodiments, the prime and the boost are administered by different routes of administration, for example, intradermal administration for one and intramuscular administration for the other.

### BRIEF DESCRIPTION OF THE DRAWINGS

FIG. 1A-B depict the characterization of SARS-CoV-2 RBD. FIG. 1A depicts multiple sequence alignment of RBDs of SARS-CoV-2, SARS-CoV, and MERS-CoV spike (S) proteins. GenBank accession numbers are QHR63250.1 (SARS-CoV-2 S), AY278488.2 (SARS-CoV S), and AFS88936.1 (MERS-CoV S). Variable residues between SARS-CoV-2 and SARS-CoV are highlighted with darker shading, and conserved residues among SARS-CoV-2, SARS-CoV, and MERS-CoV are highlighted with lighter shading. Asterisks represent fully conserved residues, colons represent highly conserved residues, and periods represent moderately conserved residues. The alignment was performed using Clustal Omega. FIG. 1B depicts SDS-PAGE (FIG. 1B, left panel) and Western blot (FIG. 1B, center and right panels) analysis of RBD-Fc fusion proteins. The protein molecular weight marker (kDa) is indicated on the left. SARS-CoV and MERS-CoV RBDs were included as controls. Antisera (1:3,000 dilution) from mice immunized with SARS-CoV RBD (FIG. 1B, center panel) and MERS-CoV RBD (FIG. 1B, right panel) were used for Western blot analysis.

FIG. 2A-E depict the detection of SARS-CoV-2 RBD binding to human ACE2 receptor. FIG. 2A depicts flow cytometry analysis of receptor expression in stable cell lines, (FIG. 2A, left panel) 293T cells alone expressed neither human ACE2 (hACE2) receptor (dashed line) nor hDPP4 receptor (solid line); (FIG. 2A, center panel) hACE2-expressing 293T (hACE2/293T) cells expressed only hACE2 (dashed line), but not hDPP4 (solid line); (FIG. 2A, right panel) hDPP4-expressing 293T (hDPP4/293T) cells expressed only hDPP4 (solid line), but not hACE2 (dashed line). Mock-incubated cells (area with hatching) were used as control. Representative images and median fluorescence intensity (MFI)±standard error (s.e.m.) were shown (n=4). FIG. 2B depicts flow cytometry analysis of SARS-CoV-2 RBD binding to cell-associated hACE2 receptor in hACE2/293T stable cell lines. SARS-CoV-2 RBD protein bound strongly to hACE2/293T cells (FIG. 2B, left panel, dashed line), but not to hDPP4/293T cells (FIG. 2B, lower left panel, broken line). SARS-CoV RBD protein bound to hACE2/293T cells (FIG. 2B, upper center panel, dashed line), but not to hDPP4/293T cells (FIG. 2B, lower center panel, broken line). MERS-CoV RBD protein did not bind to hACE2/293T cells (FIG. 2B, upper right panel, dashed line), but rather bound to hDPP4/293T cells (FIG. 2B, lower right panel, broken line). Human IgG Fc (hIgG-Fc, hereinafter hFc) protein-incubated cells (solid line) and mock-incubated cells (area with hatching) were included as controls (FIG. 2B). Representative images and MFI±s.e.m. were shown (n=4). FIG. 2C depicts immunofluorescence detection of SARS-CoV-2 RBD binding to cell-associated hACE2 receptor in hACE2/293T cells. SARS-CoV-2 RBD protein, SARS-CoV RBD protein and MERS-CoV RBD protein, each of which was fused with a C-terminal hFc, were stained with FITC-labeled goat anti-human IgG antibody (1:500) (green). hACE2 was stained with a goat-anti-hACE2 antibody (5 μg/ml) and Alexa-Fluor 647-labeled anti-goat antibody (red) (1:200). Fc-fused MERS-CoV RBD protein did not bind to hACE2, so only hACE2 (red), but not RBD (green), was detected in hACE2/293T cells. Nuclei were stained with 4′,6-diamidino-2-phenylindole (DAPI, blue). Scale bar: 10 μm. Representative images are shown. FIG. 2D depicts detection of dose-dependent binding of SARS-CoV-2 RBD to soluble hACE2 (sACE2) receptor by ELISA (FIG. 2D, left panel). The SARS-CoV-2 RBD binding to soluble hDPP4 (sDPP4) receptor (FIG. 2D, right panel), and the binding of both SARS-CoV RBD and MERS-CoV RBD to sACE2 (FIG. 2D, left panel), or sDPP4 (FIG. 2D, right panel), were tested. Control: hFc protein. Data are presented as mean A450±s.e.m. (n=4). 50% effective dose (EC50) was calculated for the binding between SARS-CoV-2 RBD ( [symbol] ) or SARS-CoV RBD ( [symbol] ) and hACE2 protein (FIG. 2D, upper panel, sACE2), or the binding between MERS-CoV RBD and hDPP4 protein (sDPP4, [symbol] ) (FIG. 2D, right panel). FIG. 2E depicts flow cytometry analysis of inhibition of SARS-CoV-2 RBD binding to hACE2/293T cells by sACE2. Binding of SARS-CoV-2 RBD to hACE2/293T cells (2E, top and lower left panels, broken line) was blocked by sACE2 (FIG. 2E, top panel, dot and dashed line), but not by sDPP4 (FIG. 2E, lower left panel, short dashed line). hFc protein-incubated cells (dotted line) and mock-incubated cells (area with hatching) were included as controls (FIG. 2E, top and middle panels). Representative images are shown. The blocking ability of sACE2 or sDPP4, as described above, was expressed as MFI±s.e.m. (n=4) (FIG. 2E, lower right panel). Low MFI correlates with high blockage. Experiments were repeated twice and yielded similar results.

FIG. 3A-C depict a comparison of SARS-CoV-2 RBD binding to human and bat ACE2 receptors. Flow cytometry analysis of SARS-CoV-2 RBD binding to hACE2 and bat ACE2 (bACE2) receptors in 293T cells transiently expressing hACE2 or bACE2. 293T cells were transiently transfected with hACE2 or bACE2 plasmid and incubated with SARS-CoV-2 RBD protein at various concentrations for analysis. SARS-CoV RBD and MERS-CoV RBD were included as controls. Representative images of SARS-CoV-2 RBD (2.5 μg/ml) binding to bACE2/293T (FIG. 3A, left panel, dot and dashed line), or hACE2/293T (FIG. 3B, left panel, dot and dashed line), cells were shown. Binding of SARS-CoV RBD (2.5 μg/ml) to bACE2/293T (FIG. 3A, right panel, dashed line), or hACE2/293T (FIG. 3B, right panel, dashed line), cells was used as a comparison. MERS-CoV RBD (broken line) and mock-incubated (area with hatching) cells (FIG. 3A-B) were included as controls. FIG. 3C depicts dose-dependent binding of SARS-CoV-2 RBD to bACE2/293T (FIG. 3C, top panel), or hACE2/293T (FIG. 3C, bottom panel), cells by flow cytometry analysis. Significant differences between binding of SARS-CoV-2 RBD ( [symbol] ) and SARS-CoV RBD ( [symbol] ) to cell-associated bACE2 receptor (FIG. 3C, top panel), or hACE2 receptor (FIG. 3C, bottom panel) were identified based on the EC50 values. The data are presented as mean±s.e.m. (n=4). Experiments were repeated twice and yielded similar results.

FIG. 4A-C depict the ability of SARS-CoV-2 RBD to inhibit viral entry, as well as its cross-reactivity and cross-neutralizing activity with SARS-CoV. FIG. 4A depicts dose-dependent inhibition of SARS-CoV-2 RBD protein against pseudotyped SARS-CoV-2 entry into hACE2/293T cells. SARS-CoV and MERS-CoV RBDs, as well hDPP4/293T cells, were included as controls. SARS-CoV-2 RBD protein inhibited entry of SARS-CoV-2 and SARS-CoV pseudoviruses into their respective target (hACE2/293T) cells (FIG. 4A, top panel), but not the entry of MERS-CoV pseudovirus into its target (hDPP4/293T) cells (FIG. 4A, top panel). SARS-CoV RBD protein inhibited both SARS-CoV-2 and SARS-CoV pseudovirus entry, but not MERS-CoV pseudovirus entry (FIG. 4A, middle panel). MERS-CoV RBD inhibited neither SARS-CoV-2 nor SARS-CoV pseudovirus entry, but it did inhibit MERS-CoV pseudovirus entry (FIG. 4A, bottom panel). The data are presented as mean inhibition (%)±s.e.m. (n=4), and 50% inhibition concentration (IC₅₀) was calculated for SARS-CoV-2 RBD (FIG. 4A, top and middle panels, [symbol] ), or SARS-CoV RBD (FIG. 4A, top and middle panels, [symbol] ), protein against SARS-CoV-2 pseudovirus and SARS-CoV pseudovirus and for MERS-CoV RBD protein ( [symbol] ) against MERS-CoV pseudovirus (FIG. 4A, bottom panel). FIG. 4B depicts cross-reactivity of SARS-CoV-2 RBD protein with SARS-CoV RBD-specific mouse sera by ELISA. Sera of mice immunized with mammalian cell-expressed SARS-CoV RBD protein were tested. Sera of mice immunized with mammalian cell-expressed MERS-CoV RBD protein were used as control. The data are presented as mean A450±s.e.m. (n=4). The IgG antibody (Ab) titers were calculated as the endpoint dilution that remains positively detectable for SARS-CoV-2 RBD ( [symbol] ), or SARS-CoV RBD ( [symbol] ), binding to anti-SARS-Coy RBD sera (FIG. 4B, top panel) and for MERS-CoV RBD ( [symbol] ) binding to anti-MERS-CoV RBD sera (FIG. 4B, bottom panel). FIG. 4C depicts cross-neutralization of SARS-CoV RBD-immunized mouse sera against SARS-CoV-2 pseudovirus entry by pseudovirus neutralization assay. MERS-Coy RBD-immunized mouse sera were used as control. The data are presented as mean neutralization (%)±s.e.m. (n=4). 50% neutralizing antibody titers (NT₅₀) were calculated against SARS-CoV-2 pseudovirus ( [symbol] ), or SARS-CoV pseudovirus ( [symbol] ), (FIG. 4C, top panel) infection in hACE2/293T target cells, as well as against MERS-CoV pseudovirus ( [symbol] ) (FIG. 4C, bottom panel) infection in hDPP4/293T cells. Experiments were repeated twice and yielded similar results.

FIG. 5A-D depict the cross-reactivity of SARS-CoV-2 RBD with SARS-CoV RBD-specific mAbs and the cross-neutralization of these mAbs against SARS-CoV-2 infection. FIG. 5A depicts cross-reactivity (binding) of SARS-CoV-2 RBD protein with SARS-CoV RBD-specific mAbs by ELISA. FIG. 5B depicts binding of these mAbs to SARS-CoV RBD protein as a comparison. SARS-CoV RBD-immunized mouse sera were included as positive control (Pos con), whereas MERS-CoV RBD-specific mAb was used as negative control (Neg con). The data are presented as mean A450±s.e.m. (n=3). FIG. 5C depicts cross-neutralization of SARS-CoV RBD-specific mAbs against SARS-CoV-2 pseudovirus infection by pseudovirus neutralization assay. FIG. 5D depicts neutralization of these mAbs against SARS-CoV pseudovirus infection was used as comparison. SARS-CoV RBD-immunized mouse sera were included as positive control (Pos con), whereas MERS-CoV RBD-specific mAb was used as negative control (Neg con). The data are presented as mean neutralization (%)±s.e.m. (n=3). Experiments were repeated twice and yielded similar results.

FIG. 6A-B depict the design of nucleoside-modified SARS-CoV-2 S1 and RBD mRNAs and detection of their expression. FIG. 6A depicts schematic map of SARS-CoV-2 S protein and construction of SARS-CoV-2 S1 and RBD mRNA vaccines. Each mRNA consists of a 5′-Cap (with the Cap 1 structure), 5′-untranslated region (UTR), tissue plasminogen activator (tPA) signal peptide with nucleoside-modified coding sequences (S1 or RBD of SARS-CoV-2), 3′-UTR, and a 3′-Poly-A tail. The mRNAs were synthesized by replacing UTP with pseudouridine (ψ) triphosphate, followed by encapsulation with lipid nanoparticles (LNPs) to form mRNA-LNPs. FIG. 6B depicts analysis of expression of SARS-CoV-2 S1 and RBD mRNA-encoding protein by Western blot. The mRNAs were transfected into 293T cells, and cell lysates and supernatants were collected 48 h post-transfection to detect protein expression using mouse sera (1:1,000 dilution) immunized with SARS-CoV-2 RBD-Fc. Mock cells were used as negative control. Protein molecular weight marker (kDa) is shown on the left.

FIG. 7 depicts the design of nucleoside-modified SARS-CoV-2 S1 and RBD mRNAs fused with an N-terminal mCherry tag. Each mRNA consists of a 5′-Cap (with the Cap 1 structure), 5′-untranslated region (UTR), a tPA signal peptide with nucleoside-modified coding sequences (mCherry and S1 or RBD of SARS-CoV-2), 3′-UTR, and a Poly-A tail. The synthesized nucleoside-modified mRNAs (containing pseudouridine (ψ) instead of uridine) were encapsulated with lipid nanoparticles (LNPs) to form mCherry-tagged SARS-CoV-2 S1 or RBD mRNA-LNPs.

FIG. 8A-B depict long-term and broad-spectrum expression of mCherry-tagged S1 and RBD mRNA-LNPs. FIG. 8A depicts the long-term expression of mCherry protein encoded by mCherry-tagged S1 and RBD mRNAs (S1-mCherry-LNP or RBD-mCherry-LNP) in 293T cells. The LNP-encapsulated mRNAs encoding SARS-CoV-2 S1 or RBD protein were incubated with 293T cells at 37° C., and the cells were then collected at different time post-incubation for analysis of mCherry signal by flow cytometry. FIG. 8B depicts the broad-spectrum expression of mCherry-tagged S1 and RBD mRNAs in different cells. The LNP-encapsulated S1 or RBD mRNA (S1-mCherry-LNP or RBD-mCherry-LNP) was incubated with each cell line at 37° C. for 48 h and analyzed for mCherry signal by flow cytometry. Data in FIGS. 8A and 8B are presented as median fluorescence intensity (MFI)±s.e.m. of triplicates (n=3). Control, empty LNPs. Experiments were repeated twice with similar results.

FIG. 9A-B depict the characterization of SARS-CoV-2 S1 and RBD mRNA-LNPs. FIG. 9A depicts the stability of LNP-encapsulated, mCherry-tagged SARS-CoV-2 S1 or RBD mRNA. The mRNAs were stored at 4° C. and 25° C. at the indicated time and then incubated with 293T cells at 37° C. for 48 h, followed by analysis for mCherry signal by flow cytometry. Data are presented as mean MFI±s.e.m. of triplicates (n=3). FIG. 9B depicts the detection of localization of mRNA-encoding protein. LNP-encapsulated, mCherry-tagged SARS-CoV-2 S1 or RBD mRNA (S1-mCherry-LNP or RBD-mCherry-LNP) was incubated with 293T cells at 37° C. for 48 h. Cell lysosome (Lyso, green) and nuclei (blue) were stained, and subcellular localization of mRNA expression based on mCherry (red) signal was analyzed by immunofluorescence microscope. Representative images are shown. Scale bar, 10 μm. Empty LNPs were used as control. Experiments were repeated twice with similar results.

FIG. 10A-C depict the induction of strong Tfh and GC B cell responses by SARS-CoV-2 RBD mRNA-LNPs. BALB/c mice were intradermally (I.D.) immunized with LNP-encapsulated SARS-CoV-2 S1 (S1-LNP) or RBD (RBD-LNP) mRNA (30 μg/mouse), or empty LNPs (control), I.D. boosted at 4 weeks, and collected for lymph nodes and spleens 10 days post-2nd immunization. Detection of T follicular helper (Tfh) (FIG. 10A), germinal center (GC) B (FIG. 10B), and plasma (FIG. 10C) cells in immunized mouse lymph nodes (for Tfh and GC B cells) and spleens (for plasma cells). Frequencies of Tfh (CD45+CD4+CD185+PD-1+), GC B (CD45⁺B220⁺CD95⁺GL-7⁺), and plasma (B220⁺CD27⁺CD138⁺) cells were analyzed by flow cytometry. Data in FIG. 10A to FIG. 10C are presented as mean±s.e.m. of mice (n=5).

FIG. 11A-C depict immunization schedules of SARS-CoV-2 S1 and RBD mRNA-LNPs. BALB/c mice were immunized with SARS-CoV-2 S1 or RBD mRNA-LNP (S1-LNP or RBD-LNP), or control (empty LNP) for three vaccination schedules. FIG. 11A depicts I.D. prime and I.D. boost immunization with 30 μg immunogens. BALB/c mice were I.D. primed and boosted with each mRNA-LNP (30 μg/mouse) or control. Ten days post-2nd immunization, mouse lymph nodes or spleens were collected to detect Tfh, GC B, plasma cells, or T cell responses, and sera were collected to detect specific antibodies, neutralizing antibodies, and inhibition of receptor binding.

FIG. 11B depicts I.D. prime and I.D. boost immunization with 10 μg immunogens. BALB/c mice were I.D. primed and boosted with each mRNA-LNP (10 μg/mouse) or control, and collected for sera at 10, 40, and 70 days post-2nd immunization to detect specific antibody responses and neutralizing antibodies. FIG. 11C depicts I.D. prime and intramuscular (I.M.) boost immunization with 10 μg immunogens. BALB/c mice were I.D. primed and I.M. boosted with each mRNA-LNP (10 μg/mouse) or control, and collected for sera at 10, 40, and 70 days post-2nd immunization to detect specific antibody responses and neutralizing antibodies.

FIG. 12A-D depict the induction of potent antibody responses and neutralizing antibodies by SARS-CoV-2 RBD mRNA-LNP vaccine. BALB/c mice were I.D. immunized with LNP-encapsulated SARS-CoV-2 S1 or RBD mRNA (S1-LNP or RBD-LNP) (30 μg/mouse), or empty LNP control, I.D. boosted at 4 weeks, and sera collected at 10 days post-2nd immunization were used for antibody detection. FIG. 12A depicts the detection of SARS-CoV-2 RBD-specific IgG antibodies by ELISA in immunized mouse sera. FIG. 12B-C depict the detection of SARS-CoV-2 RBD-specific IgG1 (FIG. 12B) and IgG2a (FIG. 12C) antibodies by ELISA in immunized mouse sera. The plates were coated with SARS-CoV-2 RBD-Fc protein, and IgG antibody (Ab) titer was calculated as the endpoint dilution that remained positively detectable. FIG. 12D depicts the detection of neutralizing antibodies against pseudotyped SARS-CoV-2 (prototype virus strain) in immunized mouse sera. A SARS-CoV-2 pseudovirus neutralization assay was used for testing. 50% neutralizing antibody titer (nAb NTH)) was calculated against SARS-CoV-2 pseudovirus infection in 293T cells expressing human ACE2 (hACE2/293T). Data are presented as mean±s.e.m. of mice (n=5).

FIG. 13A-L depict the induction of potent and long-term antibodies with neutralizing activity by SARS-CoV-2 RBD mRNA-LNP vaccine at a low immunogen dose or different routes. FIG. 13A-F depict the induction of IgG and neutralizing antibodies against infection of pseudotyped SARS-CoV-2 (prototype) at 10, 40, and 70 days, respectively, post-2nd immunization. BALB/c mice were I.D. immunized with LNP-encapsulated SARS-CoV-2 S1 or RBD mRNA (S1-LNP or RBD-LNP) vaccine (10 μg/mouse), or empty LNP (control), I.D. boosted at 4 weeks, and collected for sera at 10, 40 and 70 days post-2nd immunization to detect SARS-Coy RBD-specific IgG antibodies by ELISA (FIG. 13A, C, E) and neutralizing antibodies against infection of pseudotyped SARS-CoV-2 (prototype) (FIG. 13B, D, F). FIG. 13G-L depict the induction of IgG and neutralizing antibodies against SARS-CoV-2 (prototype) pseudovirus. BALB/c mice were I.D. immunized with LNP-encapsulated SARS-CoV-2 S1 or RBD mRNA (S1-LNP or RBD-LNP) (10 μg/mouse), or empty LNP (control), I.M. boosted at 4 weeks, and collected for sera 10 days post-2^{nd} immunization to detect SARS-CoV RBD-specific IgG antibodies by ELISA (FIG. 13G, I, K) and neutralizing antibodies against infection of pseudotyped SARS-CoV-2 (prototype) (FIG. 13H, J, L). For ELISA, the plates were coated with SARS-CoV-2 RBD-Fc protein, and IgG antibody (Ab) titer was calculated as the endpoint dilution that remained positively detectable. 50% neutralizing antibody titer (nAb NT50) was calculated against SARS-CoV-2 pseudovirus infection in hACE2/293T cells. Data in FIG. 13A to FIG. 13L are presented as mean±s.e.m. of mice (n=5). Significant differences (*, P<0.05; ** , P<0.01; ***, P<0.001) among different groups are shown in related figures.

FIG. 14A-D depict the dose-dependent inhibition of immunized mouse sera in SARS-CoV-2 RBD-hACE2 receptor binding in hACE2/293T cells by flow cytometry analysis. BALB/c mice were I.D. immunized with SARS-CoV-2 S1 or RBD mRNA-LNP vaccine (30 μg/mouse), or empty LNP control, I.D. boosted at 4 weeks, and sera collected 10 days post-2nd immunization were used for detection. Percent (%) inhibition was calculated based on relative fluorescence intensity with or without respective sera at indicated dilutions (FIG. 14A). Representative images of such inhibition by SARS-CoV-2 S1 mRNA-LNP (S1-LNP) (FIG. 14B), RBD mRNA-LNP (RBD-LNP) (FIG. 14C), or empty LNP control (FIG. 14D)-immunized mouse sera (1:5) are shown in black lines with respective MFI values. The binding between SARS-CoV-2 RBD-Fc protein (5 μg/ml) and hACE2 receptor in hACE2/293T cells is shown in grey lines. The peak filled with dark gray shading indicates Fc-hACE2/293T binding. Data are presented as mean±s.e.m. of mice (n 5).

FIG. 15A-F depict cross-reactivity of SARS-CoV-2 S1 or RBD mRNA-LNP-immunized mouse sera against SARS-CoV RBD protein. BALB/c mice were I.D. immunized with SARS-CoV-2 S1 or RBD mRNA-LNP (S1-LNP or RBD-LNP) vaccine (30 μg/mouse), or empty LNP control, I.D. boosted at 4 weeks, and sera collected at 10 days post-2nd immunization were used for detection. The plates were coated with SARS-CoV RBD-Fc protein (1 μg/ml), and serum antibodies were detected by ELISA for IgG (FIG. 15A), IgG1 (FIG. 15B), and IgG2a (FIG. 15C). The Ab titer was calculated as the endpoint dilution that remained positively detectable. FIG. 15D-15F depict the cross-neutralizing antibodies of above mouse sera against pseudotyped SARS-CoV human strains Tor2 (FIG. 15D) and GDO3 (FIG. 15E), and palm civet strain SZ3 (FIG. 15F). 50% neutralizing antibody titer (nAb NT₅₀) was calculated against infection of SARS-CoV pseudoviruses expressing S protein of Tor2, GD03, or SZ3 strain in hACE2/293T cells. Data are presented as mean±s.e.m. of mice (n=5). Significant differences (*, P<0.05; **, P<0.01; ***, P<0.001) among different groups are shown in related figures.

FIG. 16A-F depict induction of SARS-CoV-2 RBD-specific cellular immune responses by SARS-CoV-2 RBD mRNA-LNP vaccine. BALB/c mice were I.D. immunized with SARS-CoV-2 51 or RBD mRNA-LNP (S1-LNP or RBD-LNP) vaccine (30 μg/mouse), or empty LNP control, I.D. boosted at 4 weeks, and spleens collected at 10 days post-2nd immunization were analyzed for SARS-CoV-2 RBD-specific CD4+(FIG. 16A-C) and CD8+(FIG. 16D-F) T cells by flow cytometry. To determine their frequencies, IFN-γ-, TNF-α- and IL-4-producing CD45⁺CD4⁺ T cells (FIG. 16A-C) and IFN-γ-, TNF-α- and IL-4-producing CD45⁺CD8⁺ T cells (FIG. 16D-F) were stained for the corresponding cell surface marker, and intracellular cytokines. Splenocytes were incubated with a mixture of overlapping SARS-CoV-2 RBD peptides (5 μg/ml) (see Table 1). Significant differences (*, P<0.05; **, P<0.01; ***, P<0.001) among different groups are shown. Data are presented as mean±s.e.m. of mice (n=5).

FIG. 17A-I depict the induction of broad neutralizing antibodies against infection of pseudotyped SARS-CoV-2 with variant strains. SARS-CoV-2 variants containing different mutant amino acids in the spike protein are shown in each figure: 69-70del-N501Y-D614G (FIG. 17A), 69-70del-N439K-D641G (FIG. 17B), N501Y (FIG. 17C), 69-70del (FIG. 17D), V483A (FIG. 17E), E484Q (FIG. 17F), G485R (FIG. 17G), F486L (FIG. 17H), D614G (FIG. 17I). Sera of mice immunized with SARS-CoV-2 RBD mRNA-LNP (RBD-LNP) vaccine or empty LNP control were tested for their ability to neutralize infection of pseudotyped SARS-CoV-2 variants. Percent (%) neutralization was calculated against infection of pseudotyped SARS-CoV-2 in hACE2/293T cells. Data in FIG. 17A to FIG. 17I are presented as mean±s.e.m. of mouse serum samples.

### DETAILED DESCRIPTION

Development of an effective and safe vaccine against a newly recognized coronavirus, SARS-CoV-2 (also known as 2019-nCoV), is urgently needed for the prevention of current spread and future outbreaks. The present disclosure describes the development of a SARS-CoV-2 immunogenic composition based on the spike (S) protein of SARS-CoV-2. This immunogenic composition induces strong immune responses in immunized animals.

As used herein the term “immunogen” refers to any substrate that elicits an immune response in a host. As used herein an “immunogenic composition” refers to an expressed protein or a recombinant vector, with or without an adjuvant. The vector expresses and/or secretes an immunogen in vivo and the immunogen elicits an immune response in the host. The immunogenic compositions disclosed herein may or may not be immunoprotective or therapeutic. In some embodiments, the immunogenic compositions may prevent, ameliorate, palliate, or eliminate disease from the host.

A coronavirus contains four structural proteins, including spike (S), envelope (E), membrane (M), and nucleocapsid (N) proteins. Among them, S protein plays the most important roles in viral attachment, fusion and entry, and it serves as a target for development of antibodies, entry inhibitors, and vaccines. The S protein mediates viral entry into host cells by first binding to a host receptor through the receptor-binding domain (RBD) in the S1 subunit and then fusing the viral and host membranes mediated by the S2 subunit. SARS-CoV and MERS-CoV RBDs recognize different receptors. SARS-CoV recognizes angiotensin-converting enzyme 2 (ACE2), whereas MERS-CoV recognizes dipeptidyl peptidase 4 (DPP4). Similar to SARS-CoV, SARS-CoV-2 also recognizes ACE2 as its host receptor binding to viral S protein. Therefore, it is critical to define the RBD in SARS-CoV-2 S protein as the most likely target for the development of virus attachment inhibitors, neutralizing antibodies and vaccines. Full length protein sequences of the spike (S) glycoprotein and ACE2 of SARS-CoV-2 are available at the NCBI GenBank Database (Accession number: QHU79173, QHR84449, QHQ71963, QHO62107, QHO60594, QHN73795, QHD43416 and BBW89517). The following sequences are also available from the NCBI GenBank Database: SARS-CoV spike (S) proteins (Accession number: ACU31051, ACU31032, NP_828851, ABF65836, AAR91586 and AAP37017), and bat SARS-like CoV (RaTG13, AVP788042, AVP78031, ATO98231, AGZ48828, AKZ19087 and AID16716).

SEQ ID NO: 1 is a DNA sequence encoding SARS-COV-2 RBD (receptor-binding
domain) protein (residues 331-524) for transcription of SARS-COV-2 RBD
mRNA.
(SEQ ID NO: 1)
AATATCACAAACCTGTGCCCCTTTGGCGAGGTGTTCAACGCCACCCGCTTCGCCAGCGTGTATGCCTGGA
ATCGGAAGAGAATCTCCAACTGCGTGGCCGATTATTCTGTGCTGTACAACTCCGCCTCTTTCAGCACCTT
TAAGTGCTATGGCGTGTCCCCCACAAAGCTGAATGACCTGTGCTTTACCAACGTGTACGCCGATTCTTTC
GTGATCCGGGGCGACGAGGTGAGACAGATCGCCCCTGGCCAGACAGGCAAGATCGCCGATTACAATTATA
AGCTGCCAGACGATTTCACCGGCTGCGTGATCGCCTGGAACAGCAACAATCTGGACTCCAAAGTGGGCGG
CAACTACAATTATCTGTACAGGCTGTTTCGCAAGTCTAATCTGAAGCCCTTCGAGCGGGACATCTCTACA
GAAATCTACCAGGCCGGCAGCACCCCTTGCAATGGCGTGGAGGGCTTTAACTGTTATTTCCCCCTGCAGA
GCTACGGCTTTCAGCCTACCAACGGCGTGGGCTATCAGCCATACAGAGTGGTGGTGCTGAGCTTCGAGCT
GCTGCACGCACCAGCAACAGTG.
SEQ ID NO: 2 is the amino acid sequence of SARS-COV-2 RBD fragment
(residue 331-524) translated from the above DNA sequence (SEQ ID NO: 1).
(SEQ ID NO: 2)
NITNLCPFGEVFNATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDIST
EIYQAGSTPCNGVEGFNCYFPLQSYGFQPTNGVGYQPYRVVVLSFELLHAPATV
SEQ ID NO: 3 is a DNA sequence encoding SARS-COV-2 S1 protein (residues
14-660) for transcription of SARS-COV-2 S1 mRNA.
(SEQ ID NO: 3)
CAGTGCGTGAATCTGACCACAAGGACCCAGCTGCCACCTGCATACACCAACTCCTTTACAAGGGGCGTGT
ACTATCCAGATAAGGTGTTCCGCAGCTCCGTGCTGCACAGCACACAGGACCTGTTTCTGCCCTTCTTTTC
CAATGTGACCTGGTTCCACGCCATCCACGTGTCTGGCACCAACGGCACAAAGCGGTTTGATAATCCAGTG
CTGCCCTTTAACGACGGCGTGTACTTCGCCAGCACCGAGAAGTCCAATATCATCAGAGGCTGGATCTTCG
GCACCACACTGGATTCTAAGACACAGAGCCTGCTGATCGTGAACAATGCCACCAACGTGGTCATCAAGGT
GTGCGAGTTCCAGTTTTGTAACGACCCATTTCTGGGCGTGTACTATCACAAGAACAATAAGTCCTGGATG
GAGTCTGAGTTCAGAGTGTATTCTAGCGCCAACAATTGTACATTTGAGTACGTGAGCCAGCCCTTCCTGA
TGGATCTGGAGGGCAAGCAGGGCAACTTCAAGAACCTGAGGGAGTTCGTGTTTAAGAATATCGACGGCTA
CTTCAAAATCTACTCTAAGCACACCCCTATCAACCTGGTGCGCGATCTGCCACAGGGCTTTAGCGCCCTG
GAGCCTCTGGTGGACCTGCCAATCGGCATCAACATCACCAGGTTCCAGACACTGCTGGCCCTGCACCGCT
CCTACCTGACACCTGGCGATTCCTCTAGCGGATGGACCGCCGGCGCTGCCGCCTACTATGTGGGCTATCT
GCAGCCAAGGACCTTTCTGCTGAAGTACAACGAGAATGGCACCATCACAGACGCAGTGGATTGCGCCCTG
GACCCCCTGAGCGAGACCAAGTGTACACTGAAGTCCTTCACCGTGGAGAAGGGCATCTACCAGACATCCA
ATTTTCGGGTGCAGCCTACCGAGTCTATCGTGAGATTCCCAAATATCACAAACCTGTGCCCCTTTGGCGA
GGTGTTCAACGCCACCCGCTTCGCCAGCGTGTATGCCTGGAATCGGAAGAGAATCTCCAACTGCGTGGCC
GATTATTCTGTGCTGTACAACTCCGCCTCTTTCAGCACCTTTAAGTGCTATGGCGTGTCCCCCACAAAGC
TGAATGACCTGTGCTTTACCAACGTGTACGCCGATTCTTTCGTGATCCGGGGCGACGAGGTGAGACAGAT
CGCCCCTGGCCAGACAGGCAAGATCGCCGATTACAATTATAAGCTGCCAGACGATTTCACCGGCTGCGTG
ATCGCCTGGAACAGCAACAATCTGGACTCCAAAGTGGGCGGCAACTACAATTATCTGTACAGGCTGTTTC
GCAAGTCTAATCTGAAGCCCTTCGAGCGGGACATCTCTACAGAAATCTACCAGGCCGGCAGCACCCCTTG
CAATGGCGTGGAGGGCTTTAACTGTTATTTCCCCCTGCAGAGCTACGGCTTTCAGCCTACCAACGGCGTG
GGCTATCAGCCATACAGAGTGGTGGTGCTGAGCTTCGAGCTGCTGCACGCACCAGCAACAGTGTGCGGAC
CTAAGAAGTCCACCAATCTGGTGAAGAACAAGTGCGTGAACTTCAACTTCAACGGACTGACCGGCACAGG
CGTGCTGACCGAGAGCAACAAGAAGTTCCTGCCTTTTCAGCAGTTCGGCCGGGACATCGCCGATACCACA
GACGCCGTGAGAGATCCCCAGACCCTGGAGATCCTGGACATCACACCTTGCTCTTTTGGCGGCGTGAGCG
TGATCACACCCGGCACCAATACATCCAACCAGGTGGCCGTGCTGTATCAGGATGTGAATTGTACCGAGGT
GCCAGTGGCAATCCACGCAGACCAGCTGACCCCAACATGGAGGGTGTACTCCACCGGCTCTAACGTGTTC
CAGACACGCGCCGGATGTCTGATCGGAGCAGAGCACGTGAACAATTCCTAT
SEQ ID NO: 4 is the amino acid sequence of SARS-Cov-2 S protein S1
subunit (resides 14-660) translated from the above DNA sequence (SEQ
ID NO: 3).
(SEQ ID NO: 4)
QCVNLTTRTQLPPAYTNSFTRGVYYPDKVFRSSVLHSTQDLFLPFFSNVTWFHAIHVSGTNGTKRFDNPV
LPFNDGVYFASTEKSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPINLVRDLPQGFSAL
EPLVDLPIGINITRFQTLLALHRSYLTPGDSSSGWTAGAAAYYVGYLQPRTFLLKYNENGTITDAVDCAL
DPLSETKCTLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKLPDDFTGCV
IAWNSNNLDSKVGGNYNYLYRLFRKSNLKPFERDISTETYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQDVNCTEVPVAIHADQLTPTWRVYSTGSNVF
QTRAGCLIGAEHVNNSY
Certain constructs use the tissue plasminogen activator (tPA) signal
peptide amino acid sequence:
(SEQ ID NO: 5)
MDAMKRGLCCVLLLCGAVFVSAS.

TABLE 1
Overlapping peptides covering SARS-COV-2 RBD
sequences (amino acid residues 331-524)

| SEQ | | SEQ | |
|---|---|---|---|
| ID | | ID | |
| NO: | Sequence | NO: | Sequence |
| 6 | NITNLCPFGEVFNATRFASV | 7 | VFNATRFASVYAWNRKRISN |
| 8 | YAWNRKRISNCVADYSVLYN | 9 | CVADYSVLYNSASFSTFKCY |
| 10 | SASFSTFKCYGVSPTKLNDL | 11 | GVSPTKLNDLCFTNVYADSF |
| 12 | CFTNVYADSFVIRGDEVRQI | 13 | VIRGDEVRQIAPGQTGKIAD |
| 14 | APGQTGKIADYNYKLPDDFT | 15 | YNYKLPDDFTGCVIAWNSNN |
| 16 | GCVIAWNSNNLDSKVGGNYN | 17 | LDSKVGGNYNYLYRLFRKSN |
| 18 | YLYRLFRKSNLKPFERDIST | 19 | LKPFERDISTEIYQAGSTPC |
| 20 | EIYQAGSTPCNGVEGFNCYF | 21 | NGVEGFNCYFPLQSYGFQPT |
| 22 | PLQSYGFQPTNGVGYQPYRV | 23 | YQPYRVVVLSFELLHAPATV |

We identified a RBD fragment in SARS-CoV-2 S protein and found that the recombinant RBD protein bound strongly to human ACE2 (hACE2) and bat ACE2 (bACE2) receptors. In addition, the protein blocked the entry of SARS-CoV-2 and SARS-CoV into hACE2-expressing cells, suggesting that it may serve as a viral attachment inhibitor against SARS-CoV-2 and SARS-CoV infection. Moreover, we demonstrated that SARS-CoV RBD-specific polyclonal and monoclonal antibodies (mAbs) cross-reacted with SARS-CoV-2 RBD protein and inhibited SARS-CoV-2 entry into hACE2-expressing cells. We have also shown that SARS-CoV RBD-specific polyclonal antibodies and mAbs could cross-neutralize SARS-CoV-2 pseudovirus infection, supporting the potential to develop SARS-CoV RBD-based subunit vaccine for prevention of infection by SARS-CoV-2 and SARS-CoV.

We designed two LNP-encapsulated mRNA vaccines based on the S1 protein and RBD of SARS-CoV-2, respectively, and tested their stability, cell expression, immunogenicity, and neutralizing activity. Compared with the SARS-CoV-2 S1 mRNA vaccine, the capped and tailed SARS-CoV-2 RBD mRNA vaccine encapsulated in a LNP maintained stronger stability and higher translation efficiency, even under high temperatures, without losing activity, thus increasing convenience for its storage and transportation. The LNP-encapsulated mRNA encoding RBD could be efficiently expressed in a variety of human, monkey, and bat cells, indicating its capacity to reach lung and many other organs of the hosts. Different from DNA, which must be transcribed in the nucleus first, mRNA does not enter the nucleus, but can be immediately translated in the cytosol. Upon being delivered into the cell, the mRNA remains in the cytosol and is not trafficked to the lysosomes where it might be lysed by lysosomal enzymes. These features contribute to the mRNA's high stability and translation efficiency.

Germinal centers (GC) are the major sites for production of high-affinity antibodies. LNP-encapsulated SARS-CoV-2 RBD mRNA elicited Tfh and GC B cell responses and potent SARS-CoV-2 RBD-specific neutralizing antibodies able to inhibit the binding between RBD and its ACE2 receptor, demonstrating this vaccine's high potency against SARS-CoV-2. In addition, RBD mRNA-LNPs at a low dose or by different routes of administration elicited long-term and specific antibodies with potent neutralizing activity against infection by prototype and multiple mutant SARS-CoV-2 variants. The LNP-encapsulated SARS-CoV-2 RBD mRNA also induced antibodies that cross-react with SARS-CoV RBD protein and cross-neutralize SARS-CoV infection. Upon modifying the uridine nucleoside of UTP and replacing it with pseudouridine (LP), we found that the nucleoside-modified SARS-CoV-2 RBD mRNA elicited SARS-CoV-2 RBD-specific CD4⁺ and CD8⁺ T cell responses.

There are two potential advantages to the substitution of pseudouridine for uridine. One is to increase the stability of the mRNA molecule and thereby increase expression of the encoded polypeptide. The other is to avoid or reduce activation of the innate immune system, for example, through toll-like receptor (TLR) 3, TLR7 or TLR8, and thereby reduce side-effect that the mRNA might induce, separate from its intended function of inducing an antigen-specific immune response against the encoded polypeptide.

Disclosed herein is the use of mRNA as a SARS-CoV-2 vaccine. mRNA-based vaccines have a variety of advantages. Firstly, mRNA has a strong safety profile. It is active in the cytosol and does not enter the nucleus and thus does not pose a risk of integrating into chromosomal DNA, a concern with DNA-based vectors. Additionally, as a RNA vaccine it eliminates the safety concerns associated with live virus, live-attenuated virus, or viral vector-based vaccines. Furthermore, mRNA vaccines can be rapidly prepared with adequate quantity and quality to meet various vaccine manufacturing and regulatory requirements, including cost-effective manufacture; and additionally, mRNAs exhibit self-adjuvating properties without the need of separate adjuvants, capable of inducing high immune responses and simplifying the vaccination procedure. Additionally, although mRNA is often thought of as unstable as compared to protein, DNA, or whole virus vaccines, mRNA vaccines may be encapsulated with lipid nanoparticles (LNPs) for delivery, enhancing mRNA stability and preventing their degradation.

Also disclosed are immunogenic compositions. In some embodiments, the immunogenic composition comprises a protein, including, for example, immunogenic fragments of viral proteins or fusion proteins. In some embodiments, the protein is a fusion protein comprising one or more amino acid sequences encoding a SARS-CoV-2 protein or immunogenic fragment thereof. In some embodiments, the immunogenic compositions induce an immune response specific for SARS-CoV-2. In some embodiments, the immunogenic composition comprises a DNA encoding a SARS-CoV-2 protein or immunogenic fragment thereof. In some embodiments, the immunogenic composition comprises an mRNA encoding a SARS-CoV-2 protein or immunogenic fragment thereof. In some embodiments, the immunogenic composition is a protein vaccine. In some embodiments, the immunogenic composition is a DNA vaccine. In some embodiments, the immunogenic composition is an RNA vaccine.

Also disclosed are methods of preventing infection with SARS-CoV-2 by immunizing a mammal with one or more of the immunogenic compositions disclosed herein. In some embodiments, the mammal is immunized in a prime-boost scheme in which an initial, priming immunization is provided and, at a later time, one or more boosting immunizations are given. In the prime-boost scheme, each of the prime and boost immunizations can be any immunogenic composition disclosed herein. In some embodiments, the prime and boost both utilize the same immunogenic composition. In other embodiments, the prime and boost each utilize a different immunogenic composition. For example, the prime immunization can comprise an mRNA immunogenic composition and the one or more boost immunizations can include at least one protein immunogenic composition. In some embodiments, the prime immunization comprises a protein immunogenic composition, and the boost immunization comprises an mRNA immunogenic composition. Additionally, several boost immunizations can be provided to the mammal, with each individually being an mRNA or a protein immunogenic composition.

In embodiments where the immunogenic composition comprises a protein, the immunogenic composition can comprise one, two, three, or more SARS-CoV-2 proteins, or immunogenic fragments thereof, either as a mixture or in a fusion protein. In some embodiments, the one, two, three, or four SARS-CoV-2 proteins are fused to an immunopotentiator. Optionally, a trimerization stabilization sequence is disposed between the SARS-CoV-2 sequence and the immunopotentiator. In one embodiment, the stabilization sequence comprises a sequence that stabilizes the RBD protein sequence in a trimer or oligomer configuration. As used herein, the terms stabilization sequence, trimeric motif, and trimerization sequence are interchangeable and equivalent. Suitable stabilization sequences include, but are not limited to, a 27 amino acid region of the C-terminal domain of T4 fibritin (a foldon-like sequence) (GYIPEAPRDGQAYVRKDGEWVLLSTFL (SEQ ID NO: 24) or GSGYIPEAPRDGQAYVRKDGEWVLLSTFL (SEQ ID NO: 25)), a GCN4 (MKQIEDKIEEILSKIYHIENEIARIKKLIGEV (SEQ ID NO: 26)), an IQ (RMKQIEDKIEEIESKQKKIENEIARIKK (SEQ ID NO: 27)) or an IZ (IKKEIEAIKKEQEAIKKKIEAIEK (SEQ ID NO: 28)). Other suitable stabilization methods include, but are not limited to, 2,2-bipyridine-5-carboxylic acid (BPY), disulfide bonds and facile ligation.

In another embodiment, the immunopotentiator comprises a sequence to enhance the immunogenicity of the immunogenic composition. Suitable immunopotentiators include, but are not limited to, an Fc fragment of human IgG, a C3d (a complement fragment that promotes antibody formation binding to antigens enhancing their uptake by dendritic cells and B cells), an Ov ASP-1 (Onchocerca volvulus homologue of the activation associated secreted gene family) (see U.S. Pat. No. 7,700,120, which is incorporated by reference herein for all it discloses regarding ASP-1 adjuvants), a cholera toxin, a muramyl peptide, or a cytokine.

TABLE 2
Amino acid sequences of immunopotentiators

Foldon (Fd):
GYIPEAPRDGQAYVRKDGEWVLLSTFL (SEQ ID NO: 24)
human IgG Fc (hFc):
RSDKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQGNVFSCSVM
HEALHNHYTQKSLSLSPGK (SEQ ID NO: 29)
mouse IgG Fc (mFc):
RSPRGPTIKPCPPCKCPAPNLLGGPSVFIFPPKIKDVLMISLSPIVTCVVVDVSEDDPDVQISWFVNNVE
VHTAQTQTHREDYNSTLRVVSALPIQHQDWMSGKEFKCKVNNKDLPAPIERTISKPKGSVRAPQVYVLPP
PEEEMTKKQVTLTCMVTDFMPEDIYVEWTNNGKTELNYKNTEPVLDSDGSYFMYSKLRVEKKNWVERNSY
SCSVVHEGLHNHHTTKSFSRTPGK (SEQ ID NO: 30)
rabbit IgG Fc (rFc):
RSSKPTCPPPELLGGPSVFIFPPKPKDTLMISRTPEVTCVVVDVSQDDPEVQFTWYINNEQVRTARPPLR
EQQFNSTIRVVSTLPIAHQDWLRGKEFKCKVHNKALPAPIEKTISKARGQPLEPKVYTMGPPREELSSRS
VSLTCMINGFYPSDISVEWEKNGKAEDNYKTTPAVLDSDGSYFLYSKLSVPTSEWQRGDVFTCSVMHEAL
HNHYTQKSISRSPGK (SEQ ID NO: 31)
Human C3d (aa residues 1002-1303 in C3):
HLIVTPSGCGEQNMIGMTPTVIAVHYLDETEQWEKFGLEKRQGALELIKKGYTQQLAFRQPSSAFAAFVK
RAPSTWLTAYVVKVFSLAVNLIAIDSQVLCGAVKWLILEKQKPDGVFQEDAPVIHQEMIGGLRNNNEKDM
ALTAFVLISLQEAKDICEEQVNSLPGSITKAGDFLEANYMNLQRSYTVAIAGYALAQMGRLKGPLLNKFL
TTAKDKNRWEDPGKQLYNVEATSYALLALLQLKDFDFVPPVVRWLNEQRYYGGGYGSTQATFMVFQALAQ
YQKDAPDHQELNLDVSLQLPSR (SEQ ID NO: 32)
Cholera toxin b subunit (aa residues 1-124):
MTPQNITDLCAEYHNTQIHTLNDKIFSYTESLAGKREMAIITFKNGATFQVEVPGSQHIDSQKKAIERMK
DTLRIAYLTEAKVEKLCVWNNKTPRAIAAISMAN (SEQ ID NO: 33)

In one embodiment, the immunopotentiator is an immunoglobulin Fc fragment. The immunoglobulin molecule consists of two light (L) chains and two heavy (H) chains held together by disulfide bonds such that the chains form a Y shape. The base of the Y (carboxyl terminus of the heavy chain) plays a role in modulating immune cell activity. This region is called the Fc region, and is composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects including opsonization, cell lysis, and degranulation of mast cells, basophils, and eosinophils.

In certain embodiments, the SARS-CoV-2 and immunopotentiator portions of the fusion protein are linked through a flexible linker comprising (GGGGS)ₙ (SEQ ID NO: 34), wherein n is an integer between 0 and 8. In certain embodiments, n is 0, n is 1, n is 2, n is 3, n is 4, n is 5, n is 6, n is 7, or n is 8.

The disclosed SARS-CoV-2 immunogenic compositions include conservative variants of the proteins. A conservative variant refers to a peptide or protein that has at least one amino acid substituted by another amino acid, or an amino acid analog, that has at least one property similar to that of the original amino acid from an exemplary reference peptide. Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative substitution can be assessed by a variety of factors, such as, e.g., the physical properties of the amino acid being substituted (Table 3) or how the original amino acid would tolerate a substitution (Table 4). The selections of which amino acid can be substituted for another amino acid in a peptide disclosed herein are known to a person of ordinary skill in the art. A conservative variant can function in substantially the same manner as the exemplary reference peptide, and can be substituted for the exemplary reference peptide in any aspect of the present specification.

TABLE 3
Amino Acid Properties

| Property | Amino Acids |
|---|---|
| Aliphatic | G, A, I, L, M, P, V |
| Aromatic | F, H, W, Y |
| C-beta branched | I, V, T |
| Hydrophobic | C, F, I, L, M, V, W |
| Small polar | D, N, P |
| Small non-polar | A, C, G, S, T |
| Large polar | E, H, K, Q, R, W, Y |
| Large non-polar | F, I, L, M, V |
| Charged | D, E, H, K, R |
| Uncharged | C, S, T |
| Negative | D, E |
| Positive | H, K, R |
| Acidic | D, E |
| Basic | K, R |
| Amide | N, Q |

TABLE 4
Amino Acid Substitutions

| Amino | Favored | | |
|---|---|---|---|
| Acid | Substitution | Neutral Substitutions | Disfavored substitution |
| A | G, S, T | C, E, I, K, M, L, | D, F, H, N, Y, W |
| | | P, Q, R, V | |
| C | F, S, Y, W | A, H, I, M, L, T, V | D, E, G, K, N, P, Q, R |
| D | E, N | G, H, K, P, Q, R, S, T | A, C, I, L, |
| E | D, K, Q | A, H, N, P, R, S, T | C, F, G, I, L, M, V, W, |
| | | | Y |
| F | M, L, W, Y | C, I, V | A, D, E, G, H, K, N, P, |
| | | | Q, R, S, T |
| G | A, S | D, K, N, P, Q, R | C, E, F, H, I, L, M, |
| | | | T, V, W, Y |
| H | N, Y | C, D, E, K, Q, R, | A, F, G, I, L, M, P, V |
| | | S, T, W | |
| I | V, L, M | A, C, T, F, Y | D, E, G, H, K, N, P, |
| | | | Q, R, S, W |
| K | Q, E, R | A, D, G, H, M, N, | C, F, I, L, V, W, Y |
| | | P, S, T | |
| L | F, I, M, V | A, C, W, Y | D, E, G, H, K, N, P, |
| | | | Q, R, S, T |
| M | F, I, L, V | A, C, R, Q, K, T, W, | D, E, G, H, N, P, S |
| | | Y | |
| N | D, H, S | E, G, K, Q, R, T | A, C, F, I, L, M, P, |
| | | | V, W, Y |
| P | — | A, D, E, G, K, Q, | C, F, H, I, L, M, N, |
| | | R, S, T | V, W, Y |
| Q | E, K, R | A, D, G, H, M, N, | C, F, I, L, V, W, Y |
| | | P, S, T | |
| R | K, Q | A, D, E, G, H, M, | C, F, I, L, V, W, Y |
| | | N, P, S, T | |
| S | A, N, T | C, D, E, G, H, K, | F, I, L, M, V, W, Y |
| | | P, Q, R, T | |
| T | S | A, C, D, E, H, I, | F, G, L, W, Y |
| | | K, M, N, P, Q, R, V | |
| V | I, L, M | A, C, F, T, Y | D, E, G, H, K, N, P, |
| | | | Q, R, S, W |
| W | F, Y | H, L, M | A, C, D, E, G, I, K, |
| | | | N, P, Q, R, S, T, V |
| Y | F, H, W | C, I, L, M, V | A, D, E, G, K, N, P, |
| | | | Q, R, S, T |

Matthew J. Betts and Robert, B. Russell, Amino Acid Properties and Consequences of Substitutions, pp. 289-316, In Bioinformatics for Geneticists, (eds Michael R. Barnes, Ian C. Gray, Wiley, 2003).

In other aspects of this embodiment, a conservative variant of an SARS-CoV-2 immunogenic composition, a SARS-CoV-2 protein amino acid sequence, or an immunopotentiator amino acid sequence can have, for example, 1, 2, 3, 4, 5, 6, 7, 8, 9, 10, 11, 12, 13, 14, 15, or more conservative substitutions, to the amino acid sequence of the SARS-CoV-2 immunogenic compositions, SARS-CoV-2 protein, or immunopotentiator disclosed herein. In other aspects of this embodiment, a conservative variant of a SARS-CoV-2 immunogenic composition, a SARS-CoV-2 protein amino acid sequence, or an immunopotentiator amino acid sequence can be, for example, an amino acid sequence having at least 1, at least 2, at least 3, at least 4, at least 5, at least 6, at least 7, at least 8, at least 9, at least 10, at least 11, at least 12, at least 13, at least 14, or at least 15 conservative substitutions to the amino acid sequence of the SARS-CoV-2 immunogenic compositions, SARS-CoV-2 protein, or immunopotentiator disclosed herein. In yet other aspects of this embodiment, a conservative variant of an SARS-CoV-2 immunogenic composition, a SARS-CoV-2 protein amino acid sequence, or an immunopotentiator amino acid sequence can be, for example, an amino acid sequence having at most 1, at most 2, at most 3, at most 4, at most 5, at most 6, at most 7, at most 8, at most 9, at most 10, at most 11, at most 12, at most 13, at most 14, or at most 15 conservative substitutions to the amino acid sequence of the SARS-CoV-2 immunogenic compositions, SARS-CoV-2 protein, or immunopotentiator disclosed herein. In further aspects of this embodiment, a conservative variant of an SARS-CoV-2 immunogenic composition, a SARS-CoV-2 protein amino acid sequence, or an immunopotentiator amino acid sequence can be, for example, an amino acid sequence having from 1 to 15, 2 to 15, 3 to 15, 4 to 15, 5 to 15, 6 to 15, 7 to 15, 1 to 12, 2 to 12, 3 to 12, 4 to 12, 5 to 12, 6 to 12, 7 to 12, 1 to 10, 2 to 10, 3 to 10, 4 to 10, 5 to 10, 6 to 10, 7 to 10, 1 to 8, 2 to 8, 3 to 8, 4 to 8, 5 to 8, 6 to 8, 1 to 6, 2 to 6, 3 to 6, 4 to 6, 1 to 4, 2 to 4, or 1 to 3 conservative substitutions to the amino acid sequence of the SARS-CoV-2 immunogenic corn positions, SARS-CoV-2 protein, or immunopotentiator disclosed herein.

A SARS-CoV-2 immunogenic composition or protein can also comprise variants of the indicated proteins. In aspects of this embodiment, a variant of a SARS-CoV-2 immunogenic composition or protein can be, for example, an amino acid sequence having at least 75%, at least 80%, at least 85%, at least 90%, at least 95%, at least 97%, at least 98%, or at least 99% amino acid sequence identity to the SARS-CoV-2 immunogenic compositions or proteins disclosed herein. In other aspects of this embodiment, a variant of a SARS-CoV-2 immunogenic composition or protein can be, for example, an amino acid sequence having at most 75%, at most 80%, at most 85%, at most 90%, at most 95%, at most 97%, at most 98%, or at most 99% amino acid sequence identity to the SARS-CoV-2 immunogenic compositions or proteins disclosed herein.

In other embodiments, the SARS-CoV-2 S protein sequence comprises an amino acid sequence having at least 75%, at least 80%, at least 85%, at least 90%, at least 95%, at least 97%, at least 98%, or at least 99% amino acid sequence identity to the SARS-CoV-2 S amino acid sequences disclosed herein.

In still other embodiments, the immunopotentiator sequence comprises an amino acid sequence having at least 75%, at least 80%, at least 85%, at least 90%, at least 95%, at least 97%, at least 98%, or at least 99% amino acid sequence identity to the immunopotentiator amino acid sequences disclosed herein.

Expression systems such as the following are suitable for use in expressing the disclosed proteins and fusion proteins: mammalian cell expression systems such as, but not limited to, the pcDNA and GS Gene expression systems; insect cell expression systems such as, but not limited to, Bac-to-Bac, baculovirus, and DES expression systems; and E. coli expression systems including, but not limited to, pET, pSUMO, and GST expression systems.

Various advantages are associated with expression of proteins in mammalian cell expression systems. The mammalian cell expression system is a relatively mature eukaryotic system for expression of recombinant proteins. It is more likely to achieve a correctly folded soluble protein with proper glycosylation, making the expressed protein maintain its native conformation and keep sufficient bioactivity. This system can either transiently or stably express recombinant antigens, and promote signal synthesis. Recombinant proteins expressed in this way may maintain proper antigenicity and immunogenicity. However, both insect and bacterial expression systems provide inexpensive and efficient expression of proteins, which may be appropriate under certain conditions.

The purification systems used to purify the recombinant proteins are dependent on whether a tag is linked or fused with the coronavirus sequence. If the fusion proteins are fused with IgG Fc, Protein A, or Protein G, affinity chromatography is used for the purification. If the fusion proteins are fused with GST proteins, the GST columns will be used for the purification. If the fusion proteins link with 6×His tag at the N- or C-terminal, the expressed proteins are to be purified using His tag columns. If no tag is linked with the fusion protein, the expressed protein could be purified using fast protein liquid chromatography (FPLC), high performance liquid chromatography (HPLC), or other chromatography.

In certain embodiments, the immunogenic compositions further comprise or are administered with an adjuvant. Adjuvants suitable for use in animals include, but are not limited to, Freund's complete or incomplete adjuvants, Sigma Adjuvant System (SAS), and Ribi adjuvants. Adjuvants suitable for use in humans include, but are not limited to, MF59 (an oil-in-water emulsion adjuvant); Montanide ISA 51 or 720 (a mineral oil-based or metabolizable oil-based adjuvant); aluminum hydroxide, -phosphate, or -oxide; HAVLOGEN® (an acrylic acid polymer-based adjuvant, Intervet Inc., Millsboro, DE); polyacrylic acids; oil-in-water or water-in-oil emulsion based on, for example a mineral oil, such as BAYOL™ or MARCOL™ (Esso Imperial Oil Limited, Canada), monophosphoryl lipid A (MPL) (a non-toxic derivative of lipopolysaccharide (LPS) and a toll-like receptor (TLR) agonist), or a vegetable oil such as vitamin E acetate; saponins; and Onchocerca volvulus activation-associated protein-1 (Ov ASP-1) (see U.S. Pat. No. 7,700,120, which is incorporated by reference herein for all it discloses regarding Ov ASP-1 adjuvants). However, components with adjuvant activity are widely known and, generally, any adjuvant may be utilized that does not adversely interfere with the efficacy or safety of the vaccine and/or immunogenic composition.

Vaccines and/or immunogenic compositions according to the various embodiments disclosed herein can be prepared and/or marketed in the form of a liquid, frozen suspension, or in a lyophilized form. Typically, vaccines and/or immunogenic compositions prepared according to the present disclosure contain a pharmaceutically acceptable carrier or diluent customarily used for such compositions. Carriers include, but are not limited to, stabilizers, preservatives, and buffers. Suitable stabilizers are, for example SPGA, Tween compositions (such as are available from A.G. Scientific, Inc., San Diego, CA), carbohydrates (such as sorbitol, mannitol, starch, sucrose, dextran, glutamate, or glucose), proteins (such as dried milk serum, albumin, or casein), or degradation products thereof. Examples of suitable buffers include alkali metal phosphates. Suitable preservatives include thimerosal, merthiolate, and gentamicin. Diluents include water, aqueous buffer (such as buffered saline), alcohols, and polyols (such as glycerol).

Also disclosed herein are methods for inducing an immune response to a SARS-CoV-2 using the disclosed proteins, nucleic acids, or immunogenic compositions. Generally, the vaccine and/or immunogenic composition may be administered subcutaneously, intradermally, submucosally, intranasally, or intramuscularly in an effective amount to prevent infection from the SARS-CoV-2 and/or treat an infection from the SARS-CoV-2. An effective amount to prevent infection is an amount of immunizing protein, encoding nucleic acid, or immunogenic composition that will induce immunity in the immunized animals against challenge by a virulent virus such that infection is prevented or the severity is reduced. Immunity is defined herein as the induction of a significantly higher level of protection in a subject after immunization compared to an unimmunized group. An effective amount to treat an infection is an amount of immunizing protein, nucleic acid, or immunogenic composition that induces an appropriate immune response against SARS-CoV-2 such that severity of the infection is reduced.

Protective immune responses can include humoral immune responses and cellular immune responses. Protection against SARS-CoV-2 is believed to be conferred through serum antibodies (humoral immune response) directed to the surface proteins, with mucosal IgA antibodies and cell-mediated immune responses also playing a role. Cellular immune responses are useful in protection against SARS-CoV-2 virus infection with CD4⁺ and CD8⁺ T cell responses being particularly important. CD8⁺ immunity is of particular importance in killing virally infected cells.

Additionally, the disclosed proteins and/or immunogenic compositions can be administered using immunization schemes known by persons of ordinary skill in the art to induce protective immune responses. These include a single immunization or multiple immunizations in a prime-boost strategy. A boosting immunization can be administered at a time after the initial, prime, immunization that is days, weeks, months, or even years after the prime immunization. In certain embodiments, a boost immunization is administered 2 weeks, 3 weeks, 4 weeks, 1 month, 2, months, 3 months, 4 months, 5 months, or 6 months or more after the initial prime immunization. Additional multiple boost immunizations can be administered such as weekly, every other week, monthly, every other month, every third month, or more. In other embodiments, the boost immunization is administered every 3 weeks, every 4 weeks, every 5 weeks, every 6 weeks, every 7 weeks, every 8 weeks, every 9 weeks, every 10 weeks, every 11 weeks, or every 12 weeks. In certain embodiments, boosting immunizations can continue until a protective anti-SARS-CoV-2 antibody titer is detected in the subject's serum. In certain embodiments, a subject is administered one boost immunization, two boost immunizations, three boost immunizations, or four or more boost immunizations, as needed to obtain a protective antibody titer.

Protective immunity can be long lasting but may eventually wane with the passage of years or decades. In some embodiments, one or more boost immunizations are administered in a time interval within weeks or months of the prime immunization to establish a protective immunity, and a re-boost immunization is administered years later. In some embodiments, the re-boost immunization is administered about 1, 2, 3, 4, 5, 6, 7, 8, 9, or 10 years after the initial boost immunization(s). In some embodiments, a re-boost immunization is administered periodically, for example, about every 1, 2, 3, 4, 5, 6, 7, 8, 9, or 10 years after the initial boost immunization(s). In various embodiments, the re-boost is administered within 1, 2, 3, 4, 5, 6, 7, 8, 9, 10, or 11 months before or after the anniversary of the initial boost immunization(s), or the most recent (re-)boost immunization.

In some embodiments, the adjuvant in the initial prime immunization and the adjuvant in the boost immunizations are the same. In some embodiments, the adjuvant in the initial prime immunization and the adjuvant in the boost immunizations are different.

Further, in various formulations of the proteins, mRNAs or other nucleic acids, and/or immunogenic compositions, suitable carriers, excipients, stabilizers, and the like may be added as are known by persons of ordinary skill in the art.

The disclosed proteins, immunogenic compositions, and methods may be used to prevent or treat SARS-CoV-2 virus infection in a subject susceptible thereto such as, but not limited to, a human, a primate, a, mammal, a bird, a domesticated animal, or an animal in the wild.

### Example 1

### SARS-CoV-2 RBD Protein is an Important Target to Develop Safe and Effective COVID-19 Subunit Vaccines

### Materials and Methods

Construction, expression and purification of recombinant protein. The construction, expression and purification of recombinant RBD proteins of SARS-CoV-2, SARS-Coy, and MERS-CoV was accomplished as follows. Briefly, DNA polynucleotides encoding residues 331-524 of SARS-CoV-2 S protein, residues 318-510 of SARS-CoV S protein, or residues 377-588 of MERS-CoV S proteins, were amplified by PCR using codon-optimized DNA sequences encoding SARS-CoV-2 S protein (GenBank accession number: QHR63250.1), SARS-Coy S protein (GenBank accession number: AY278488.2), or MERS-CoV S protein (GenBank accession number: AFS88936.1), as respective templates, and ligated into pFUSE-hIgG1-Fc2 expression vector (hereinafter named hFc) to encode RBD-Fc fusion proteins. The RBD-Fc fusion proteins were expressed in human embryonic kidney (HEK)293T cells, secreted into cell culture supernatants, and purified by protein A affinity chromatography. These fusion proteins were used as the RBD proteins in the experiments described below.

SDS-PAGE and Western blot. The purified RBD-Fc fusion proteins were analyzed by SDS-PAGE and Western blot. Briefly, proteins were separated by 10% Tris-glycine SDS-PAGE and stained with Coomassie brilliant blue or transferred to nitrocellulose membranes. The blots were blocked with 5% fat-free milk in PBS containing 0.5% Tween-20 (PBST) for 2 h at 37° C. and further incubated with SARS-CoV RBD-specific polyclonal antibody (mouse sera, 1:3,000), or MERS-CoV RBD-specific antibody (mouse sera, 1:3,000), overnight at 4° C. The blots were then incubated with horseradish peroxidase (HRP)-conjugated goat anti-mouse IgG (1:5,000) for 1 h at room temperature and then visualized with ECL Western blot substrate reagents and Amersham Hyperfilm.

Flow cytometry analysis. Flow cytometry analysis was performed to detect the binding of SARS-CoV-2 RBD protein to hACE2 receptor in 293T cells stably expressing hACE2 (hACE2/293T). SARS-CoV and MERS-CoV RBDs, as well as 293T cells stably expressing hDPP4 receptor (hDPP4/293T), were used as controls. Briefly, cells were incubated with respective RBD of SARS-CoV-2, SARS-CoV, or MERS-CoV containing a C-terminal hFc at 20 μg/ml for 30 min at room temperature, which was followed by incubation with FITC-labeled goat anti-human IgG antibody (1:500) for 30 min and analyzed by flow cytometry. The blockage of RBD-receptor binding was performed by incubation of soluble human ACE2 (sACE2; 5 μg/ml) receptor with respective RBD of SARS-CoV-2, SARS-CoV, or MERS-CoV (20 μg/ml), followed by the same procedure as that described above. hIgG-Fc protein (hFc: 20 μg/ml), or soluble human DPP4 (sDPP4; 5 μg/ml) receptor, was included as control.

Detection of hACE2 protein expression in hACE2/293T, or hDPP4 protein expression in hDPP4/293T, stable cell lines was performed by flow cytometry analysis, as described above, except that the cells were sequentially incubated with hACE2- or hDPP4-specific goat antibody (0.5 μg/ml) at room temperature for 20 min and FITC-labeled anti-goat IgG antibody (1:200) for 1 h at 4° C.

Flow cytometry analysis was also performed to detect the binding between SARS-CoV-2 RBD and hACE2 or bat-ACE2 (bACE2) receptor in transiently transfected 293T cells. Briefly, 293T cells were transfected with hACE2- or bACE2-expressing plasmid using the calcium phosphate method, and 48 h later, they were incubated with SARS-CoV-2 RBD protein at various concentrations for 30 min at room temperature. SARS-CoV and MERS-CoV RBDs were included as controls. After staining with FITC-conjugated goat anti-human IgG antibody (1:500), the mixture was analyzed by flow cytometry as described above.

Immunofluorescence staining. This was performed to detect the binding between SARS-CoV-2 RBD and hACE2 receptor in hACE2/293T stable cell lines. SARS-CoV and MERS-CoV RBDs were used as controls. Briefly, cells were sequentially incubated with Fc-fused SARS-CoV-2, SARS-CoV, or MERS-CoV RBD (20 μg/ml) and hACE2-specific goat antibody (5 μg/ml) for 30 min at room temperature. After three washes, the cells were incubated with FITC-labeled goat anti-human IgG (Fc) antibody (1:500), or Alexa-Fluor 647-labeled anti-goat antibody (1:200 dilution) for 30 min at room temperature. The nuclei were stained with 4′,6-diamidino-2-phenylindole (DAPI) for 5 min and mounted in VectaMount Permanent Mounting Medium. The samples were imaged on a confocal microscope (Zeiss LSM 880), and the images were prepared using the ZEN software.

ELISA. ELISA was performed to detect the binding of SARS-CoV-2 RBD protein to sACE2 receptor. SARS-CoV and MERS-CoV RBDs, as well as sDPP4 protein, were used as controls. Briefly, ELISA plates were precoated with SARS-CoV-2, SARS-CoV, or MERS-CoV RBD (1 μg/ml) overnight at 4° C. and blocked with 2% fat-free milk in PBST for 2 h at 37° C. Serially diluted sACE2, or sDPP4, protein was added to the plates and incubated for 2 h at 37° C. After four washes, the bound protein was detected using hACE2- or hDPP4-specific goat antibody (0.5 μg/ml) for 2 h at 37° C., followed by incubation with HRP-conjugated anti-goat IgG antibody (1:5,000) for 1 h at 37° C. The reaction was visualized by addition of substrate 3,3′,5,5′-Tetramethylbenzidine (TMB) and stopped by H₂SO₄ (1N). The absorbance at 450 nm (A450) was measured by an ELISA plate reader (Tecan).

The cross-reactivity of SARS-CoV-2 RBD protein to SARS-CoV RBD-specific antibody was assessed by coating ELISA plates with either SARS-CoV-2 RBD (1 μg/ml), as well as SARS-CoV RBD or MERS-CoV RBD (as controls, 1 μg/ml), followed by sequential incubation with serially diluted SARS-CoV RBD- or MERS-CoV RBD-immunized mouse sera or RBD-specific mAbs and HRP-conjugated anti-mouse IgG (1:5,000) antibodies.

Pseudovirus neutralization and inhibition assays. SARS-CoV-2 pseudovirus was generated as described below. Briefly, 293T cells were co-transfected with a plasmid encoding Env-defective, luciferase-expressing HIV-1 genome (pNL4-3.luc.RE) and a plasmid encoding SARS-CoV-2 S protein using the calcium phosphate method. SARS-CoV and MERS-CoV pseudoviruses were packaged as controls. The transfected medium was changed into fresh Dulbecco's Modified Eagle's Medium (DMEM) 8 h later, and pseudovirus-containing supernatants were collected 72 h later for single-cycle infection in target cells. Pseudovirus neutralization assay was then performed by incubation of SARS-CoV-2, SARS-CoV, or MERS-CoV pseudovirus with serially diluted SARS-CoV RBD- or MERS-CoV RBD-immunized mouse sera (controls), or RBD-specific mAbs, for 1 h at 37° C., followed by addition of the mixture into hACE2/293T (for SARS-CoV-2 pseudovirus and SARS-CoV pseudovirus) or hDPP4/293T (for MERS-CoV pseudovirus) target cells. It should be noted that the SARS-CoV or MERS-CoV RBD-immunized mouse serum controls were not generated with the RBD-Fc fusion proteins described herein. Fresh medium was added 24 h later, and the cells were lysed 72 h later in cell lysis buffer. The lysed cell supernatants were incubated with luciferase substrate and detected for relative luciferase activity using the Infinite 200 PRO Luminator (Tecan). The 50% MERS pseudovirus neutralizing antibody titer (NT50) was calculated using the CalcuSyn computer program.

Inhibition of pseudovirus entry by SARS-CoV-2 RBD protein was performed as described below. Briefly, SARS-CoV-2 RBD protein at serial dilutions was incubated with hACE2/293T target cells for 1 h at 37° C. After removing medium containing the protein, the cells were infected with SARS-CoV-2 pseudovirus. SARS-CoV RBD and MERS-CoV RBD, as well as SARS-CoV pseudovirus and MERS-CoV pseudovirus, were used as controls. Fresh medium was added 24 h later, and the cells were lysed and analyzed, as described above. The 50% inhibitory concentration (IC₅₀) of the RBD protein was calculated using the CalcuSyn computer program.

Statistical analysis. Values were expressed as mean and standard error (s.e.m). Statistical significance between different groups was calculated by GraphPad Prism Statistical Software. Two-tailed Student's t-test was used. *** represents P<0.001.

Results

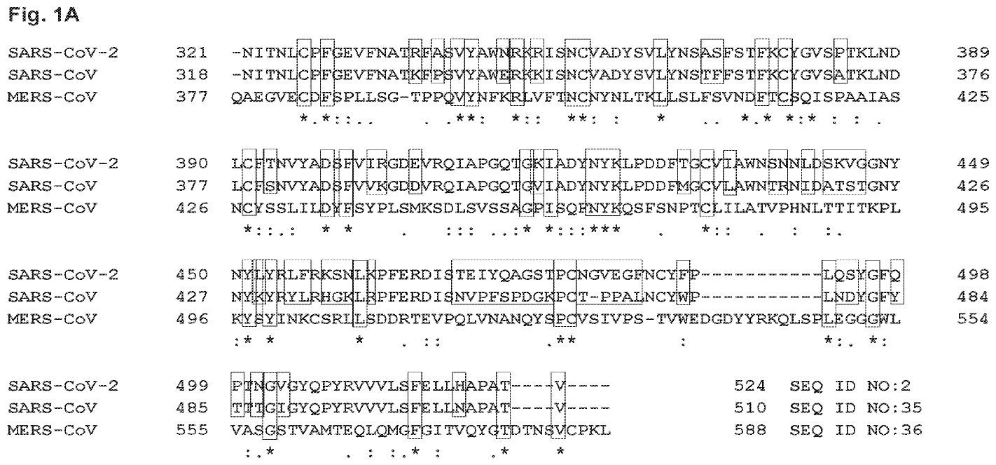

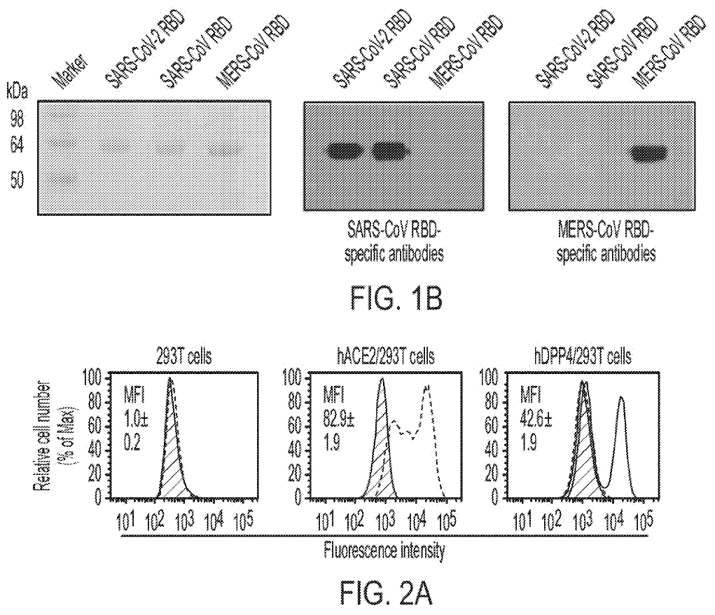

By alignment of the RBD sequences of SARS-CoV and SARS-CoV-2, we identified the region of SARS-CoV-2 RBD as residues 331 to 524 of S protein (FIG. 1A). We then constructed a recombinant RBD protein containing codon-optimized RBD sequences with a C-terminal Fc of human IgG1 (hFc) using pFUSE-hIgG1-Fc2 expression vector, expressed the protein in mammalian 293T cells, and purified the fusion protein from cell culture supernatants using protein A affinity chromatography. Similar to prior experience with SARS-CoV and MERS-CoV RBD protein controls, SARS-CoV-2 RBD protein had high expression with high purity (FIG. 1B, left panel). Notably, only SARS-CoV-2 and SARS-CoV RBDs were recognized by SARS-CoV RBD-specific polyclonal antibodies, but not MERS-CoV RBD-specific polyclonal antibodies (FIG. 1B, center panel). Conversely, only MERS-CoV RBD was recognized by MERS-CoV RBD-immunized polyclonal antibodies (FIG. 1B, right panel), indicating cross-reactivity of SARS-CoV-2 RBD with SARS-CoV RBD-specific antibodies, but not with MERS-CoV RBD-specific antibodies.

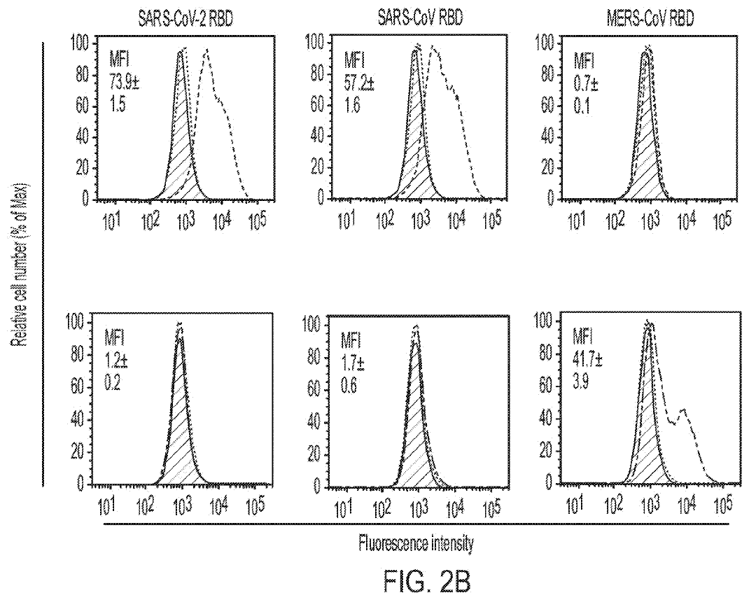

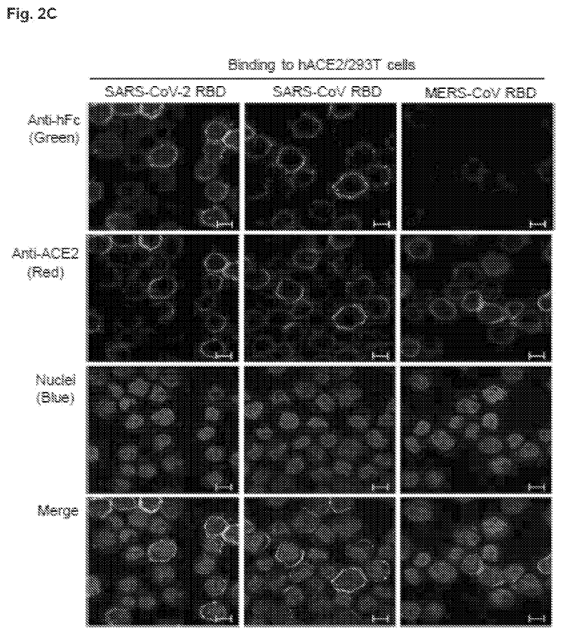

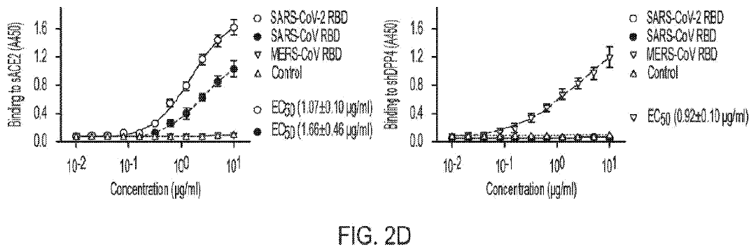

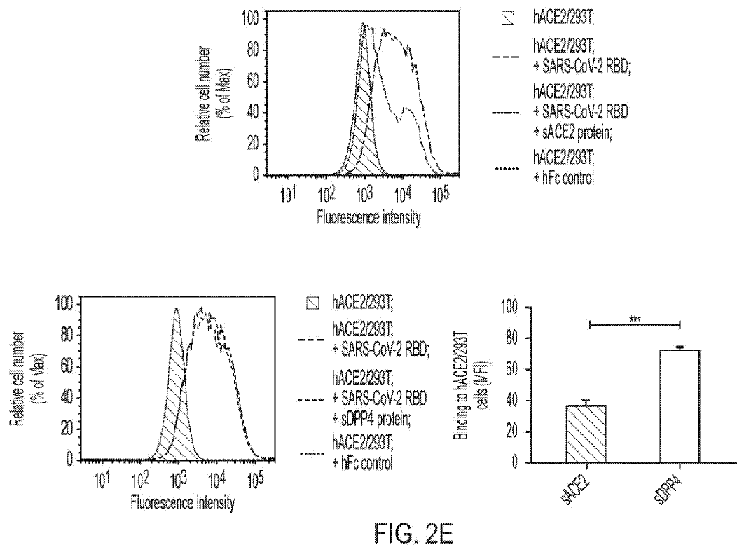

Four experiments were performed to detect binding between SARS-CoV-2 RBD and the hACE2 receptor. First, we tested if stably transfected hACE2/293T cells expressed hACE2 by flow cytometry analysis. Since 293T cells alone did not express either hACE2 or hDPP4, they could not be recognized by anti-hACE2 or anti-hDPP4 antibodies (FIG. 2A, left panel). Only hACE2/293T cells, but not hDPP4/293T cells, expressed hACE2, which was recognized by an anti-hACE2 antibody (FIG. 2A, center panel), whereas only hDPP4/293T cells, but not hACE2/293T cells, expressed hDPP4 and was, correspondingly, recognized by anti-hDPP4 antibody (FIG. 2A, right panel). These data confirmed the expression of hACE2 in hACE2/293T cells and the expression of hDPP4 in hDPP4/293T cells.

Second, we used these hACE2/293T cells to detect the binding of SARS-CoV-2 RBD protein to cell-associated hACE2 by flow cytometry analysis and immunofluorescence staining. Similar to SARS-CoV RBD, SARS-CoV-2 RBD bound to hACE2/293T cells expressing hACE2 (FIG. 2B, upper center and left panels, respectively), but not to hDPP4/293T cells expressing hDPP4 (FIG. 2B, lower enter and left panels, respectively). Furthermore, the binding between SARS-CoV-2 RBD and hACE2-expressing 293T cells was much stronger than the binding between SARS-CoV RBD and hACE2-expressing 293T cells (FIG. 2B, upper center and left panels) with the SARS-CoV-2 RBD producing a median fluorescent intensity (MFI) of 73.9±1.5 as compared to an MFI of 57.2±1.6 for the SARS-CoV RBD. MERS-CoV RBD did not bind to hACE2-expressing 293T cells (FIG. 2B, upper right panel), but rather bound to hDPP4-expressing 293T cells (FIG. 2B, lower right panel). The results from immunofluorescence staining revealed positive signals for both hACE2 and hFc on hACE2/293T cells treated with SARS-CoV-2 RBD and SARS-CoV RBD, both of which contained a C-terminal hFc tag, whereas hACE2/293T cells treated with MERS-CoV RBD (containing a C-terminal hFc tag) showed positive signals for hACE2, but not for hFc, indicating that there is no binding of MERS-CoV RBD to the hACE2-expressing cells (FIG. 2C). These data suggest that SARS-CoV-2 RBD and SARS-CoV RBD can bind to cell-associated hACE2, but not to hDPP4.

Third, we detected the binding of plate-adsorbed SARS-CoV-2 RBD to soluble hACE2 protein (sACE2) by ELISA. The results indicated that SARS-CoV-2 RBD bound to sACE2 in a dose-dependent manner and that the binding between SARS-CoV-2 RBD and sACE2 with 50% effective dose (EC₅₀) of 1.07 μg/ml was stronger than that between SARS-CoV RBD and sACE2 (EC₅₀: 1.66 μg/ml). In contrast, MERS-CoV RBD did not bind to sACE2 (FIG. 2D, upper panel). While neither SARS-CoV-2 RBD nor SARS-CoV RBD bound to sDPP4, MERS-CoV RBD strongly bound to sDPP4 (EC₅₀: 0.92 μg/ml) (FIG. 2D, lower panel). These data indicate that both SARS-CoV-2 RBD and SARS-CoV RBD could bind to hACE2 in solution, but not to hDPP4 in solution.

Fourth, flow cytometry analysis further indicated that the binding between SARS-CoV-2 RBD and cell-associated hACE2 receptor could be significantly blocked by sACE2 protein (FIG. 2E, top and bottom panels), but not by sDPP4 protein (FIG. 2E, middle and bottom panels). Taken together, the above results confirm that the identified SARS-CoV-2 RBD could bind to both cell-associated and soluble hACE2 proteins.

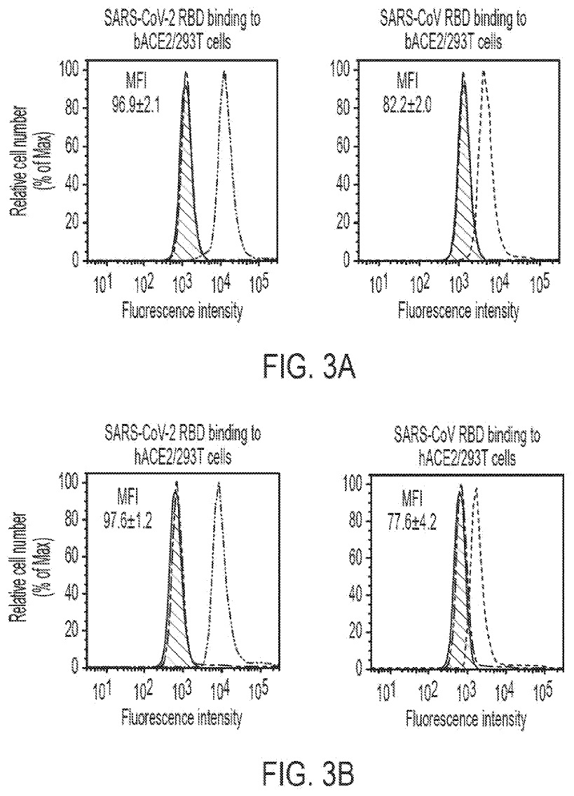

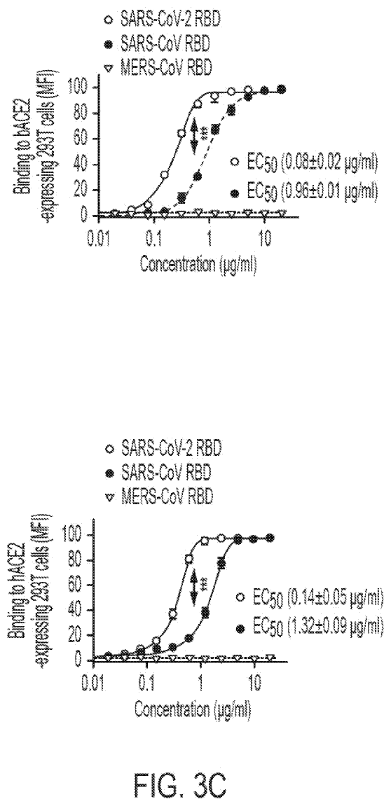

Like SARS-CoV and MERS-CoV, SARS-CoV-2 is believed to originate from bats. Next, we determined the binding affinity of the identified SARS-CoV-2 RBD to bat ACE2 (bACE2) and compared this binding with that of SARS-CoV RBD. We transiently transfected a bACE2-expressing plasmid into 293T cells and included a hACE2-expressing plasmid as a control, followed by detection of fluorescence intensity 48 h later. Results indicated that SARS-CoV-2 RBD bound strongly to 293T-expressed bACE2 with intensity similar to that of its binding to 293T-expressed hACE2 (FIG. 3A, left panel and 3B, left panel, respectively), and that this binding occurred in a dose-dependent manner (FIG. 3C). In addition, the binding affinity between SARS-CoV-2 RBD and 293T-expressed bACE2 (EC₅₀: 0.08 μg/ml) or hACE2 (EC₅₀: 0.14 μg/ml) was significantly higher than that between SARS-CoV RBD and 293T-expressed bACE2 (EC₅₀: 0.96 μg/ml) or hACE2 (EC₅₀: 1.32 μg/ml) (FIG. 3A-C). Nevertheless, MERS-CoV RBD bound neither bACE2- nor hACE2-expressing 293T cells (FIG. 3C). These data indicate that SARS-CoV-2 RBD can bind to both bACE2 and hACE2 with significantly stronger binding than that of SARS-CoV RBD to either bACE2 or hACE2, supporting the bat origin of SARS-CoV-2. These results may partially explain why SARS-CoV-2 is more transmissible than SARS-CoV.

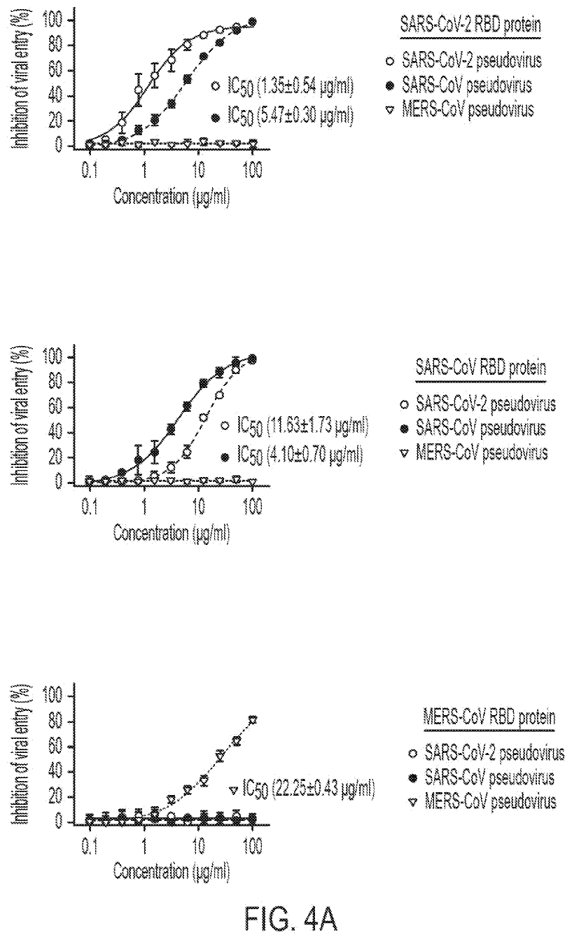

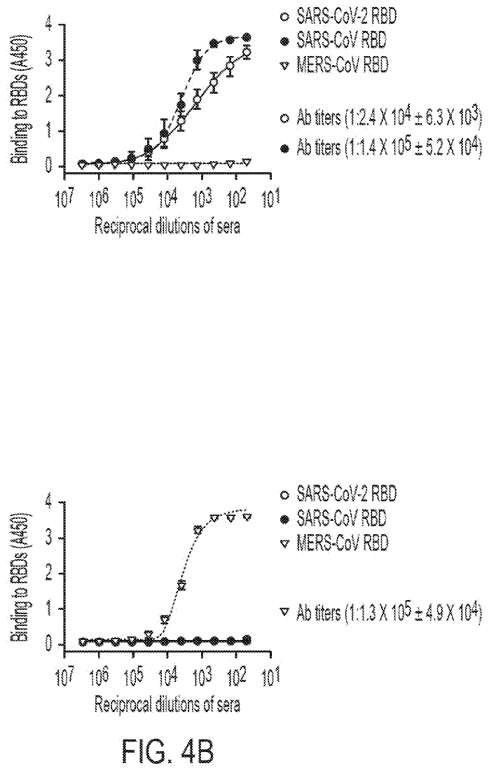

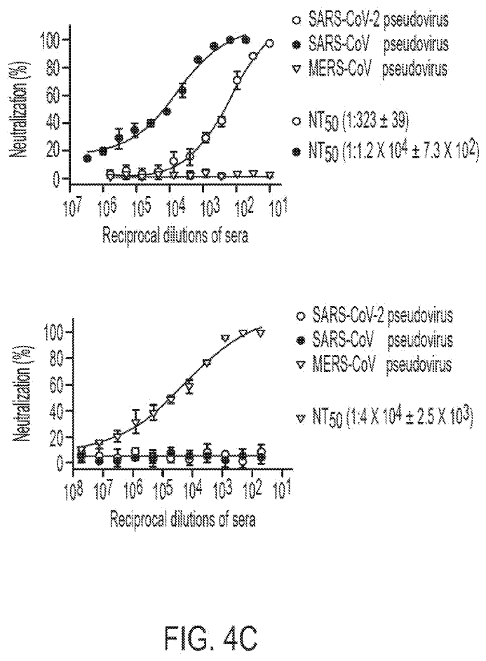

We then evaluated the potential of the identified SARS-CoV-2 RBD protein as an inhibitor of viral entry. To accomplish this, we first generated a pseudotyped SARS-CoV-2 by co-transfection of a plasmid encoding Env-defective, luciferase-expressing HIV-1 (pNL4-3.luc.RE) and a plasmid expressing S protein of SARS-CoV-2 into 293T cells, followed by collection of pseudovirus-containing supernatants. We then incubated serially diluted SARS-CoV-2 RBD protein with hACE2/293T target cells, followed by the addition of pseudovirus and detection of inhibitory activity of infection. With the capacity for only one-cycle infection, S protein-expressing pseudovirus itself cannot replicate in the target cells. Therefore, the inhibition of pseudovirus infection represents inhibition of viral entry, as mediated by viral S protein. As expected, SARS-CoV-2 RBD protein inhibited SARS-CoV-2 pseudovirus entry into hACE2-expressing 293T cells in a dose-dependent manner with 50% inhibition concentration (IC₅₀) as low as 1.35 μg/ml. Interestingly, it also blocked the entry of SARS-CoV pseudovirus into hACE2-expressing 293T cells with IC₅₀ of 5.47 μg/ml (FIG. 4A, left panel). Similarly, SARS-CoV RBD protein blocked the entry of both SARS-CoV pseudovirus and SARS-CoV-2 pseudovirus into hACE2-expressing 293T cells with 1050 of 4.1 and 11.63 μg/ml, respectively (FIG. 4A, center panel). In addition, neither SARS-CoV-2 RBD nor SARS-CoV RBD blocked the entry of MERS-CoV pseudovirus into hDPP4-expressing 293T cells (FIG. 4A, right and center panels). MERS-CoV RBD did not block the entry of SARS-CoV-2 pseudovirus or SARS-CoV pseudovirus into hACE2-expressing 293T cells, but it did block the entry of MERS-CoV pseudovirus into hDPP4-expressing 293T cells (IC₅₀: 22.25 μg/ml) (FIG. 4A, right panel). These results indicate that SARS-CoV-2 RBD protein could be developed as an effective therapeutic agent against SARS-CoV-2 and SARS-CoV infection.

Since SARS-CoV-2 is more phylogenetically related to SARS-CoV than MERS-CoV, we further assessed the cross-reactivity of SARS-CoV RBD-specific antibodies with SARS-CoV-2 RBD and cross-neutralizing activity of SARS-CoV RBD-specific antibodies against pseudotyped SARS-CoV-2 in a further four experiments. First, we performed an ELISA to detect the cross-reactivity of SARS-CoV RBD-immunized mouse sera with SARS-CoV-2 RBD. The results showed that SARS-CoV-2 RBD reacted strongly with anti-SARS-CoV RBD IgG with antibody titer of 1:2.4×10⁴ (FIG. 4B, left panel), but it did not react with anti-MERS-CoV RBD IgG antibody (FIG. 4B, right panel). As expected, SARS-CoV RBD reacted strongly with anti-SARS-CoV RBD IgG (antibody titer: 1:1.4×10⁵) (FIG. 4B, left panel), but not with anti-MERS-CoV RBD IgG antibody (FIG. 4B, right panel). MERS-CoV RBD did not react with anti-SARS-CoV RBD IgG, but instead reacted with anti-MERS-CoV RBD IgG (antibody titer: 1:1.3×10⁵) (FIG. 4B).

Second, we performed a pseudovirus neutralization assay to detect the cross-neutralizing activity of SARS-CoV RBD-immunized mouse sera against SARS-CoV-2 pseudovirus infection. Results revealed that SARS-CoV RBD-specific antisera could neutralize SARS-CoV-2 pseudovirus infection with a neutralizing antibody titer of 1:323, while these antisera could neutralize SARS-CoV pseudovirus infection with higher neutralizing antibody titer (1:1.2×10⁴) (FIG. 4C, top panel). MERS-CoV RBD-inducing mouse sera only neutralized MERS-Coy pseudovirus infection in hDPP4-expressing cells with a neutralizing antibody titer of 1:4×10⁴ (FIG. 4C, bottom panel), but failed to neutralize infection by either SARS-CoV-2 pseudovirus or SARS-CoV pseudovirus (FIG. 4C, top panel). These data indicate that SARS-CoV RBD-specific polyclonal antibodies can cross-react with SARS-CoV-2 RBD and cross-neutralize SARS-CoV-2 pseudovirus infection.

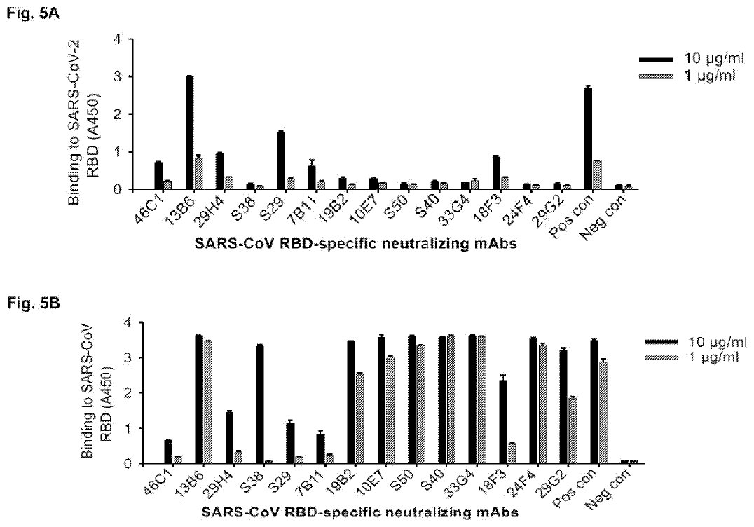

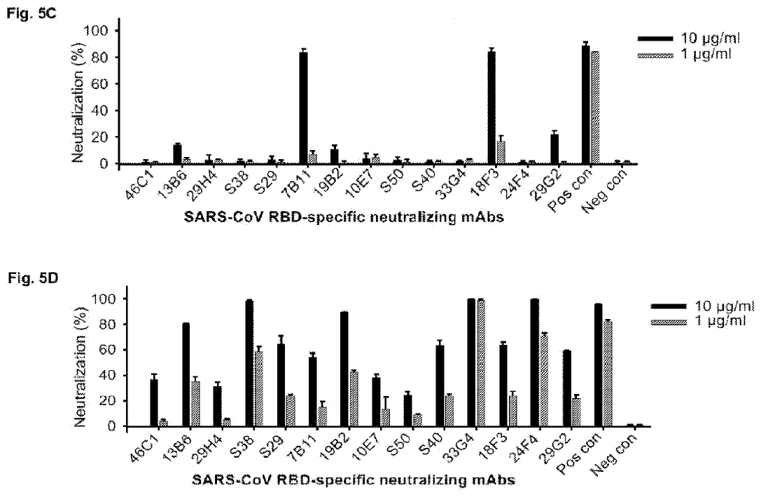

Third, we further performed an ELISA to detect the cross-reactivity of a series of SARS-CoV RBD-specific mAbs with SARS-CoV-2 RBD. The results demonstrated that among the mAbs tested, several mAbs, including 46C1, 29H4, 7B11, 18F3, S29, and 13B6, bound SARS-CoV-2 RBD protein in a dose-dependent manner, with higher binding at 10 μg/ml than 1 μg/ml (FIG. 5A), although all of these mAbs can bind SARS-CoV RBD protein, most having strong binding affinity at 10 and 1 μg/ml (FIG. 5B). In contrast, MERS-CoV RBD-specific mAb control had no reactivity with SARS-CoV-2 and SARS-CoV RBD proteins (FIG. 5A-B, Neg con).

Fourth, we carried out the pseudovirus neutralization assay to detect the cross-neutralizing ability of these SARS-CoV RBD-specific mAbs against SARS-CoV-2 pseudovirus infection. Results indicated that two mAbs (7B11 and 18F3) could neutralize SARS-CoV-2 pseudovirus infection with about 80% neutralization at 10 μg/ml (FIG. 5C). As expected, all these mAbs neutralized SARS-CoV pseudovirus infection, most of which had >50% neutralizing activity at 10 μg/ml, and a few reached >50% neutralization at 1 μg/ml (FIG. 5D). Nevertheless, MERS-Coy RBD-specific mAb control had no cross-neutralizing activity against both SARS-CoV-2 and SARS-CoV pseudoviruses (FIG. 5C-D, Neg con). These data suggest that SARS-CoV RBD-specific mAbs can cross-react with SARS-CoV-2 RBD and cross-neutralized infection of SARS-CoV-2 pseudovirus.

In summary, we have characterized the SARS-CoV-2 RBD which exhibits strong binding to its cell-associated and soluble ACE2 receptors with human and bat origin. SARS-CoV-2 RBD protein could block S protein-mediated SARS-CoV-2 pseudovirus and SARS-CoV pseudovirus entry into ACE2 receptor-expressing target cells, indicating that SARS-CoV-2 RBD protein can act as a viral attachment or entry inhibitor against SARS-CoV-2 and SARS-CoV. SARS-CoV RBD-induced polyclonal and monoclonal antibodies could cross-react with SARS-CoV-2 RBD and cross-neutralize SARS-CoV-2 pseudovirus infection, indicating that SARS-CoV RBD-specific antibodies may be used for treatment of SARS-CoV-2 infection and that either SARS-CoV RBD protein or SARS-CoV-2 RBD protein may be used as a vaccine to induce neutralizing or cross-neutralizing antibodies for prevention or amelioration of SARS-CoV-2 or SARS-CoV infection.

### Example 2

### SARS-CoV-2 RBD-Based mRNA Vaccine is Effective in Inducing Highly Potent Neutralizing Antibodies Against Prototype and Multiple Variant Strains of SARS-CoV-2

### Materials and Methods

Design and synthesis of nucleoside-modified SARS-CoV-2 S1 and RBD mRNAs. SARS-CoV-2 S1 or RBD mRNAs with modified nucleosides were constructed as follows. Briefly, genes encoding S1 (residues 14-660) and RBD (residues 331-524) of SARS-CoV-2 S protein were amplified using PCR and codon-optimized SARS-CoV-2 S plasmid (GenBank accession number QHR63250.1) as template. The amplified S1 or RBD (without mCherry tag) genes contained N-terminal T7 promotor, 5′-untranslated region (5′-UTR), tPA signal peptide and C-terminal 3′-UTR, and they were inserted into pCAGGS-mCherry vector. To construct N-terminal mCherry-tagged S1 or RBD mRNAs, the above genes were amplified and fused to the C-terminal mCherry of this vector.

Synthesis of nucleoside-modified SARS-CoV-2 S1 and RBD mRNAs. The mRNAs were synthesized, 5-capped and 3′ tailed with poly-A. Briefly, the above S1 or RBD genes were linearized using BgI II enzyme and synthesized using the MEGAscript® T7 Kit, in the presence of CTP, ATP, GTP, and pseudouridine-5′-triphosphate (pseudouridine, ψ) (to replace uridine triphosphate (UTP) in order to increase stability and expression of target antigens). The synthesized and purified mRNAs were capped using Cap 1 Capping System Kit to produce the Cap 1 structure and tailed with Poly(A) Polymerase Tailing Kit to obtain a Poly-A tail of about 150 base pair (bp).

Preparation of SARS-CoV-2 S1- or RBD mRNA-LNPs. The lipid mixture (ethanol phase) was used to encapsulate PNI Formulation Buffer (Precision NanoSystems, Inc.; aqueous phase) containing SARS-CoV S1 or RBD mRNA (0.174 mg/ml) at 1:3 ratio (ethanol:aqueous, V/V). The LNP-encapsulated mRNAs were diluted in PBS, filtered through a 0.22-mm filter, and concentrated using Amicon Ultra Centrifugal Filters. The empty LNP control was prepared using PNI Formulation Buffer without mRNAs as aqueous phase.

mRNA transfection and protein expression. Unencapsulated mRNAs were transfected into 293T cells using TransIT®-mRNA Kit (Mirus Bio). Briefly, SARS-CoV-2 S1 or RBD mRNA (1 μg) was mixed with TransIT®-mRNA and boost reagents in Opti-Minimal Essential Medium (MEM). The mixture was added to cells containing complete DMEM medium and cultured at 37° C. with 5% CO₂. 72 h after transfection, supernatant was collected, and cells were lysed in RIPA buffer for detection of protein expression by Western blot. Samples were incubated with 4× Laemmli buffer, separated on a 10% polyacrylamide gel, and transferred to PVDF membrane, which was blocked with 5% fat-free milk in PBS containing 0.5% Tween-20 (PBST). S1 and RBD protein expression was detected by sequential incubation of the membrane with mouse sera (1:1,000) immunized with SARS-CoV-2 RBD-Fc protein and horseradish peroxidase (HRP)-conjugated goat anti-mouse IgG-Fab (1:5,000) for 1 h at room temperature. The signals were detected using ECL Western blot substrate reagents and Amersham Hyperfilm.

Flow cytometry. Flow cytometry was used to detect the expression of LNP-encapsulated SARS-CoV-2 S1 or RBD mRNA in different cells. Briefly, human cell lines, including A549 (human lung), Hep-2 (human respiratory tract), HEP-G2 (human liver), Caco-2 (human intestinal tract), HeLa (human genitourinary tract), 293T (human kidney), African green monkey kidney cells (Vero E6), and bat lung cells (Tb1-Lu), were pre-plated into 24-well culture plates (2×10⁵/well) containing complete DMEM 24 h before experiments. The cells were then incubated with mCherry-tagged SARS-CoV-2 51 or RBD mRNA-LNP (1 μg/ml) and cultured at 37° C. 48 h later or at indicated time points, the cells were collected for analysis of mCherry signal by flow cytometry.

Immunofluorescence staining. This was performed to detect localization of mRNA-coding S1 or RBD protein. Briefly, mCherry-tagged SARS-CoV-2 S1 or RBD mRNA-LNPs (1 μg/ml) were added to 293T cells (2×10⁵/well) pre-plated 24 h before experiments; the cells were cultured at 37° C. for 48 h and harvested for immunofluorescence staining. The cells were then fixed and permed with FIX and PERM Cell Permeabilization Kit, followed by incubation with FITC-labeled anti-human CD107a (LAMP-1, for lysosome) antibody (1:100) for 30 min at room temperature. After washing with PBS, the concentrated cell suspension was evenly distributed into slides, counter-stained with DAPI (4′,6-diamidino-2-phenylindole, 300 nM) for nuclei for 5 min, and then mounted in VectaMount Permanent Mounting Medium. The slides were imaged on a confocal microscope (Zeiss LSM 880). Images were prepared using ZEN software.

Thermal stability of mRNA-LNPs. The stability of LNP-encapsulated SARS-CoV-2 S1 or RBD mRNA was performed as described below. Briefly, mCherry-tagged SARS-CoV-2 S1 or RBD mRNA-LNPs were stored at 4° C. and 25° C. for 0, 24, and 72 h and then added to 293T cells at a concentration of 1 μg/ml. The cells were cultured at 37° C. for 48 h and analyzed for mCherry signal by flow cytometry.

Animals. Six-to-eight-week-old male and female BALB/c mice were used in the study. The animal protocols were approved by the Institutional Animal Care and Use Committee (IACUC) of New York Blood Center). All animal studies were carried out in strict accordance with the guidance and recommendations in the Guide for the Care and Use of Laboratory Animals (National Research Council Committee).

Mouse immunization and sample collection. Three immunization protocols were performed. First, mice were immunized by intradermal (I.D.) injection with SARS-CoV-2 S1 or RBD mRNA-LNP (30 μg/100 μl/mouse), or empty LNP (control), and I.D. boosted at 4 weeks with the same immunogens (I.D.-I.D.). Ten days after the 2nd immunization, mouse lymph nodes were collected to detect T follicular helper (Tfh) and germinal center (GC) B cells, sera were collected to detect antibody response (production), neutralizing antibodies (activity), and inhibition of receptor binding, and spleens were collected to detect plasma cells and SARS-CoV-2-specific T cell responses, as described below.

Second, mice were I.D. immunized with SARS-CoV-2 S1 or RBD mRNA-LNP (10 μg/100 μl/mouse), or empty LNP (control), and I.D. boosted at 4 weeks with the same immunogens (I.D.-I.D.). Sera were collected at 10, 40, and 70 days post-2nd immunization, and assessed for antibody response and neutralizing antibodies.

Third, mice were I.D. immunized with SARS-CoV-2 S1 or RBD mRNA-LNP (10 μg/100 μl/mouse), or empty LNP (control), and intramuscularly (I.M.) boosted at 4 weeks with the same immunogens (I.D.-I.M.). Sera were collected at 10, 40, and 70 days post-2nd immunization, and assessed for antibody responses and neutralizing antibodies.

ELISA. ELISA was performed to detect SARS-CoV-2 or SARS-CoV RBD-specific antibodies in immunized mouse sera. Briefly, ELISA plates were coated with SARS-CoV-2 or SARS-CoV RBD-Fc protein (1 μg/ml) overnight at 4° C. and blocked with 2% fat-free milk in PBST for 2 h at 37° C. After three washes with PBST, the plates were sequentially incubated with serially diluted mouse sera and HRP-conjugated anti-mouse IgG (1:5,000), IgG1 (1:5,000), or IgG2a (1:2,000) antibodies for 1 h at 37° C. The plates were sequentially incubated with substrate TMB (3,3′,5,5′-tetramethylbenzidine), and H₂SO₄ (1N) was used to stop the reaction. Absorbance at 450 nm was measured using an ELISA plate reader.

Pseudovirus neutralization assay. mRNA-LNP vaccine-induced neutralizing antibodies against SARS-CoV-2 and SARS-CoV pseudovirus infection were detected using a pseudovirus neutralization assay. Briefly, to obtain pseudovirus, 293T cells were co-transfected with a plasmid encoding S protein of wild-type (GenBank accession number QHR63250.1) or variant strains of SARS-CoV-2, as well as SARS-CoV Tor2 strain (GenBank accession number AY274119) and a plasmid encoding Env-defective, luciferase-expressing HIV-1 genome (pNL4-3.luc.RE). SARS-CoV GDO3 (GDO3T0013, GenBank accession number AY525636) or SARS-CoV SZ3 (GenBank accession number AY304486) pseudoviruses were prepared as described above except for using Tor2 S protein-encoding plasmid containing mutations for GDO3 and SZ3 at their respective RBD regions. Culture supernatants containing pseudoviruses were collected at 72 h after transfection, incubated with serially diluted mouse sera for 1 h at 37° C., added to hACE2/293T cells, and then cultured at 37° C. The cells were lysed using cell lysis buffer 72 h post-culture and transferred into luminometer plates. Luciferase substrate was added to the plates and the reaction mixture was assayed for relative luciferase activity using the Infinite 200 PRO Luminometer. Neutralizing activity of serum antibodies against SARS-CoV-2 and SARS-CoV pseudoviruses was calculated and expressed as 50% pseudovirus neutralizing antibody titer (NT50), or percent neutralization (%).

Inhibition of binding of SARS-CoV-2 RBD protein to hACE2 receptor. Flow cytometry was used to analyze the ability of immunized mouse sera to block the binding between SARS-CoV-2 RBD protein and cell-associated hACE2 receptor. Briefly, hACE2/293T cells were incubated with SARS-CoV-2 RBD-Fc protein (5 μg/ml) in the presence or absence of serially diluted mouse sera for 30 min at room temperature. The cells were stained with FITC-labeled goat anti-human IgG-Fc antibody (1:500) for 30 min at room temperature and measured for fluorescence by flow cytometry.

Isolation and analysis of lymph node cells. Lymph nodes collected at 10 days post-2^{nd} immunization (I.D.) with SARS-CoV-2 mRNA-LNPs or empty LNPs were pooled for detection. Briefly, lymph nodes were homogenized into single cell suspensions in complete DMEM and filtered through a cell strainer (70 μm). The isolated cells were washed, resuspended in PBS containing 2% FBS, and stained with Fixable Viability Dye eFluor™ 780 for live and dead cells. The cells were then stained with fluorescence-labeled antibody cocktails, including anti-mouse CD45-AF700, CD4-PE/Cy7, CD185-BV605, PD-1-BV421, B220-PerCP/Cy5.5, CD95-BV510, CD138-PE, and GL-7-APC, and incubated in dark for 20 min at room temperature. The stained cells were washed, resuspended in cell-staining buffer, and analyzed for Tfh and GC B cells using flow cytometry. The data were analyzed using FlowJo software.

Plasma cell, surface and intracellular staining. Splenocytes collected at 10 days post-2^{nd} immunization (I.D.) with SARS-CoV-2 mRNA-LNPs or empty LNPs were detected for plasma cell and RBD-specific T cell immune responses. Splenocytes from homogenized spleens were resuspended in complete DMEM. Splenocytes were treated with 1× Red Blood Cell Lysis Buffer, washed with PBS, and resuspended in complete DMEM. To detect plasma cells, splenocytes (1×10⁶) were stained with a cocktail of antibodies including anti-mouse CD45-AF700, CD27-BV421, B220-PerCP/Cy5.5, and CD138-PE in Cell Staining Buffer. To detect RBD-specific T cell responses, splenocytes (1×10⁶) were incubated with a mixture of overlapping SARS-CoV-2 RBD peptides (5 μg/mL) (Table 1) and cultured at 37° C. for 72 h. At 68 h post-stimulation, 1× Brefeldin A was added to the cells. After stimulation, the cells were washed with PBS and stained with Fixable Viability Dye eFluor™ 780 for live and dead cells. The cells were stained for surface markers using anti-mouse CD45-AF700, CD4-FITC, and CD8-PerCP/Cy5.5 antibodies. After fixation and permeabilization, the cells were stained for intracellular cytokine markers using IFN-γ-PE, TNF-α-BV421, and IL-4-BV711. The stained cells were measured using flow cytometry, and the data were analyzed using FlowJo software.

Statistical analyses. All values are presented as mean plus standard error of the mean (s.e.m). Statistical differences among SARS-CoV-2 S1, RBD mRNA-LNPs, and control groups were performed using Student's two-tailed t-test. * (P<0.05), ** (P<0.01), and *** (P<0.001) represent significance and high significance among different groups. All statistical analyses were performed using GraphPad Prism 5 statistical software.

### Results

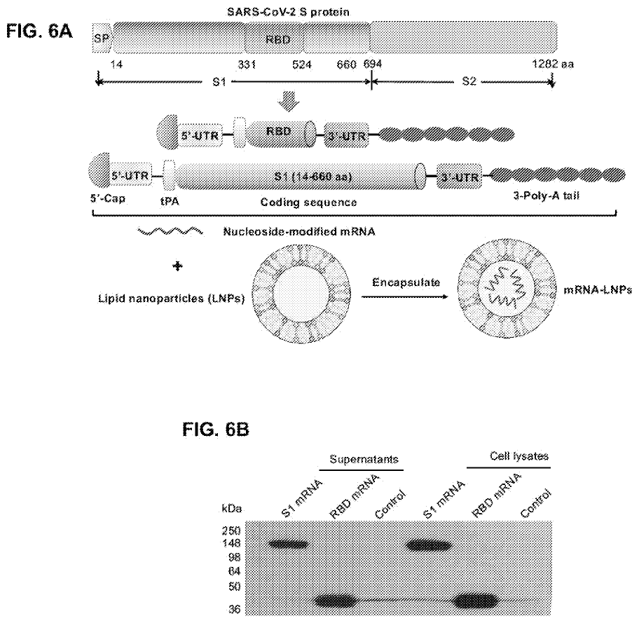

To identify an mRNA vaccine that induces strong immune responses against SARS-CoV-2, we initially designed two mRNA constructs that respectively express S1 (residues 14-660) and RBD (residues 331-524) of SARS-CoV-2 S protein (FIG. 6A). The mRNAs were synthesized, replacing uridine with pseudouridine (4)) in order to increase stability and expression of the target antigen. The mRNAs were capped and tailed with 5′-ScriptCap Cap 1 Capping System (to produce the Cap 1 structure) and 3′-Poly-A tail (about 150 bp), and then encapsulated in LNP to further increase stability (FIG. 6A). The synthesized mRNAs were tested for protein expression by Western blot. Results showed that both the culture supernatant and lysate of cells transfected with S1 or RBD mRNA, but not those of control cells, reacted strongly with a polyclonal antibody specific to SARS-CoV-2 RBD (FIG. 6B), demonstrating expression of the target proteins.

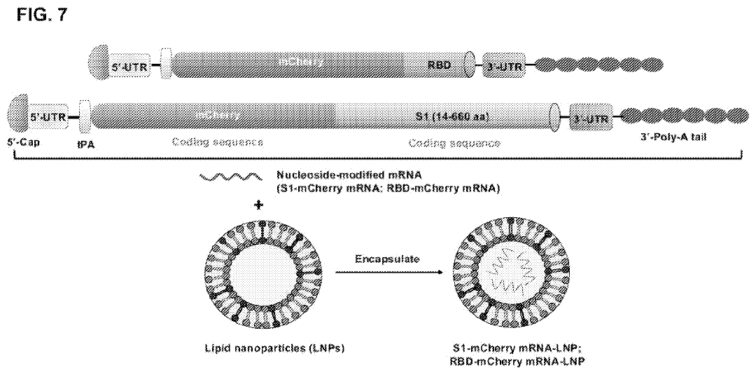

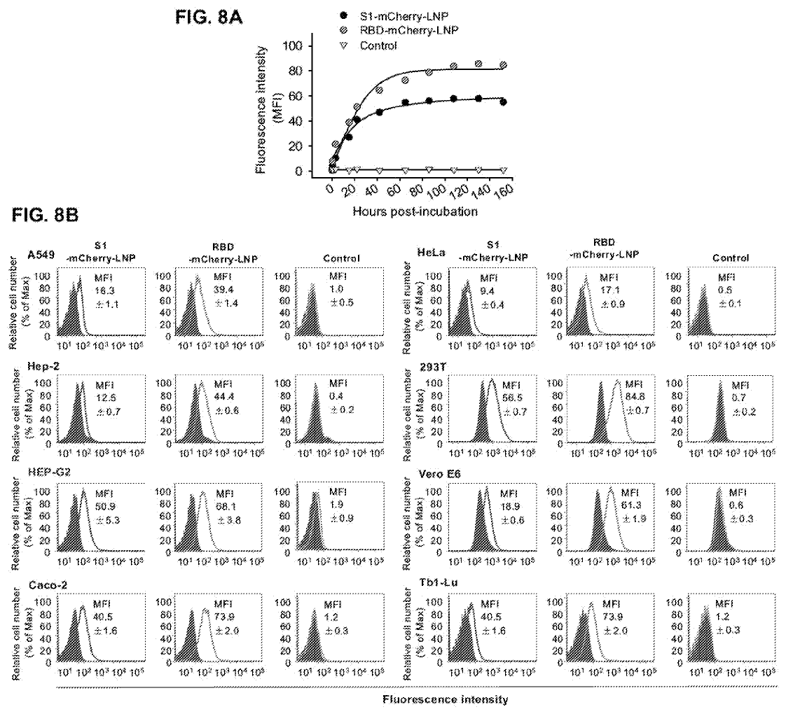

To detect the longevity of S1 and RBD mRNAs in expressing target proteins and their ability to express proteins in different cell types, we constructed N-terminal mCherry-tagged SARS-CoV-2 S1 and RBD mRNAs, encapsulated them in LNP (FIG. 7), and tested mCherry expression by flow cytometry analysis. Different from the control, both RBD- and S1-mCherry mRNAs showed expression in transfected cells for at least 160 hours during the detection period and that the RBD-mCherry mRNA conferred higher expression than the S1-mCherry mRNA (FIG. 8A). In addition, these mRNAs expressed proteins efficiently in a variety of cell lines in humans, monkeys, and/or bats, including A549 (human lung), Hep-2 (human respiratory tract), HEP-G2 (human liver), Caco-2 (human intestinal tract), HeLa (human genitourinary tract), 293T (human kidney), Vero E6 (African green monkey kidney), and Tb1-Lu (bat lung) cells (FIG. 8B). Particularly, the expression of RBD-mCherry protein was much stronger than the expression of S1-mCherry protein in all cells tested (FIG. 8B). Together, these data suggest long-term and broad expression of mRNA-encoding proteins, particularly RBD, in target cells.

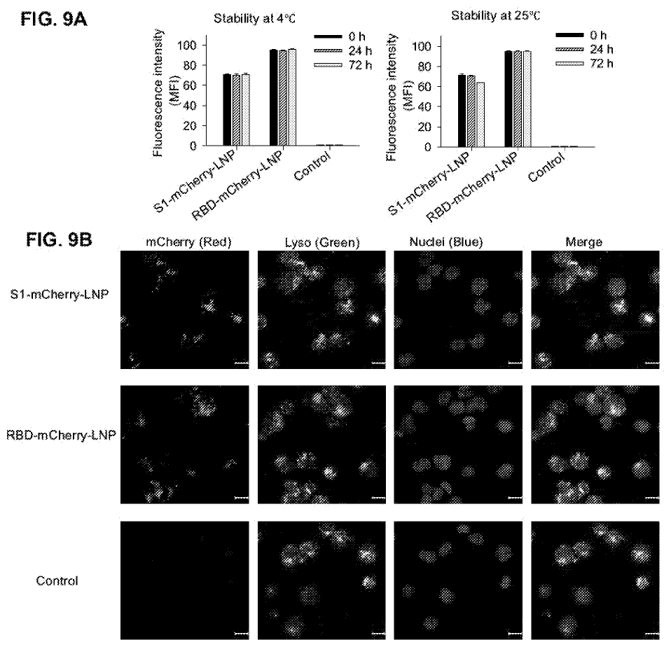

We characterized the LNP-encapsulated S1 and RBD mRNAs for their stability and localization in target cells. The mCherry-tagged S1 or RBD mRNA-LNP had median fluorescence intensity (MFI) values, as indicated by mCherry expression, after being stored at 4° C. for 0, 24, and 72 h before transfection, similar to those at 25° C. for 0, 24 and 72 h, but RBD mRNA-LNP had higher mCherry expression than that of S1 mRNA-LNP in all conditions tested (FIG. 9A). The mCherry in S1 or RBD mRNA-LNP proteins was not co-localized with nuclei and was not degraded in lysosomes (FIG. 9B). Together, these data indicate that LNP-encapsulated SARS-CoV-2 S1, particularly RBD, mRNA vaccines were stable at various temperatures and were not heavily trafficked to lysosomes and therefore resistant to degradation.

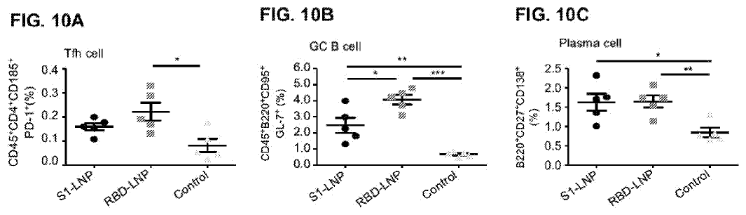

We evaluated Tfh, GC B, and plasma cell responses induced by LNP-encapsulated SARS-CoV-2 S1 and RBD mRNAs in BALB/c mice. As such, mice were I.D. immunized with each mRNA-LNPs (30 μg/mouse) or LNP control, I.D. boosted with same mRNA-LNPs, and draining lymph nodes or spleens were collected 10 days after the 2nd immunization to test Tfh, GC B, and plasma cells. Flow cytometry analysis showed that more Tfh (CD45+CD4+CD185+PD-1+) (FIG. 10A), or significantly more GC B (CD45+B220+CD95+GL-7+) (FIG. 10B), cells were detected in the lymph nodes of mice immunized with RBD mRNA-LNPs than those immunized with S1 mRNA-LNPs, whereas only a background level of Tfh or GC B cells was shown in the mice injected with control LNPs. Plasma cells were also increased in vaccinated mouse splenocytes, as compared to the control LNP group (FIG. 100). Together, these data demonstrate the recruitment of Tfh, GC B, and/or plasma cells in vivo, particularly after immunization with SARS-CoV-2 RBD mRNA-LNP vaccine.

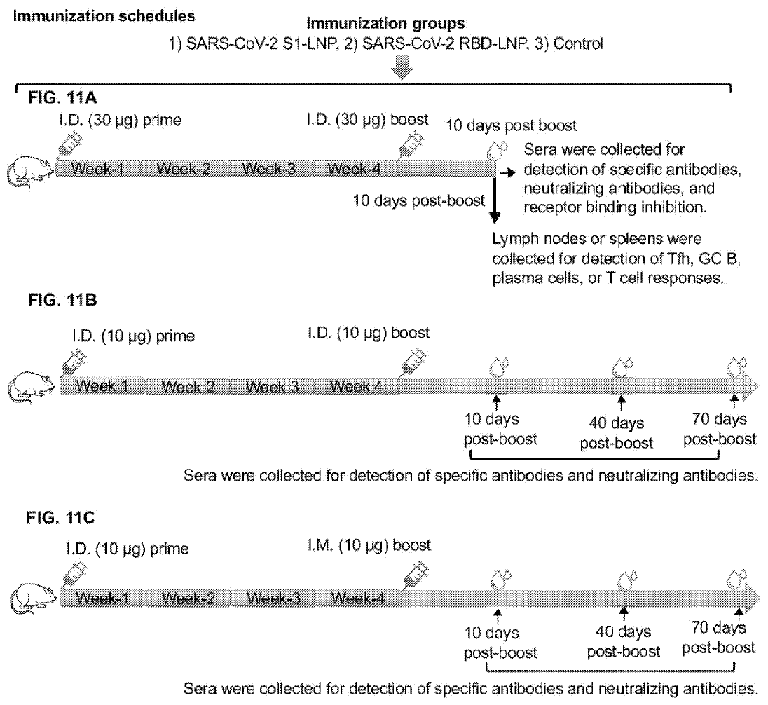

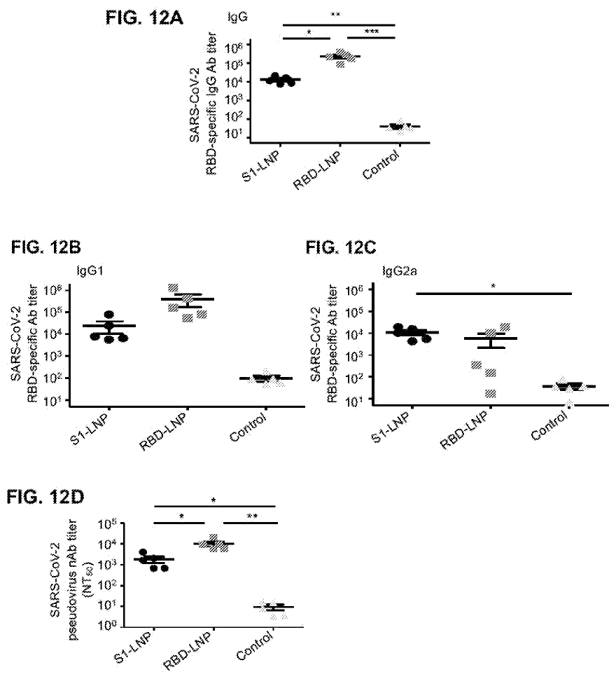

We next evaluated humoral immune responses and neutralizing antibodies induced by LNP-encapsulated SARS-CoV-2 S1 and RBD mRNAs. As such, mice were immunized with each mRNA-LNP at three different schedules (FIG. 11), and sera were collected for detection of IgG and neutralizing antibodies. First, ELISA results revealed that both S1 and RBD mRNA-LNPs at 30 μg via I.D. prime and boost induced SARS-CoV-2 RBD-specific IgG (FIG. 12A), subtype IgG1 (Th2) (FIG. 12B), and IgG2a (Th1) (FIG. 12C) antibodies 10 days after boost immunization and that IgG antibody titer induced by RBD was significantly higher than that by S1 (FIG. 12A). Pseudovirus neutralization assay showed that S1 and RBD mRNA-LNPs elicited neutralizing antibodies against SARS-CoV-2 pseudovirus entry into human ACE2-expressing 293T (hACE2/293T) cells. RBD elicited a significantly higher titer of neutralizing antibodies than S1 against SARS-CoV-2 pseudovirus infection (FIG. 12D).

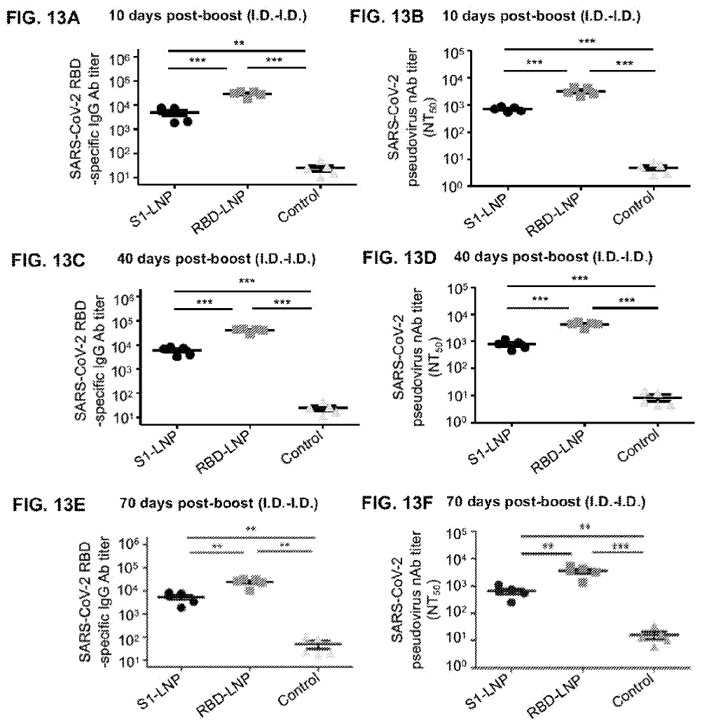

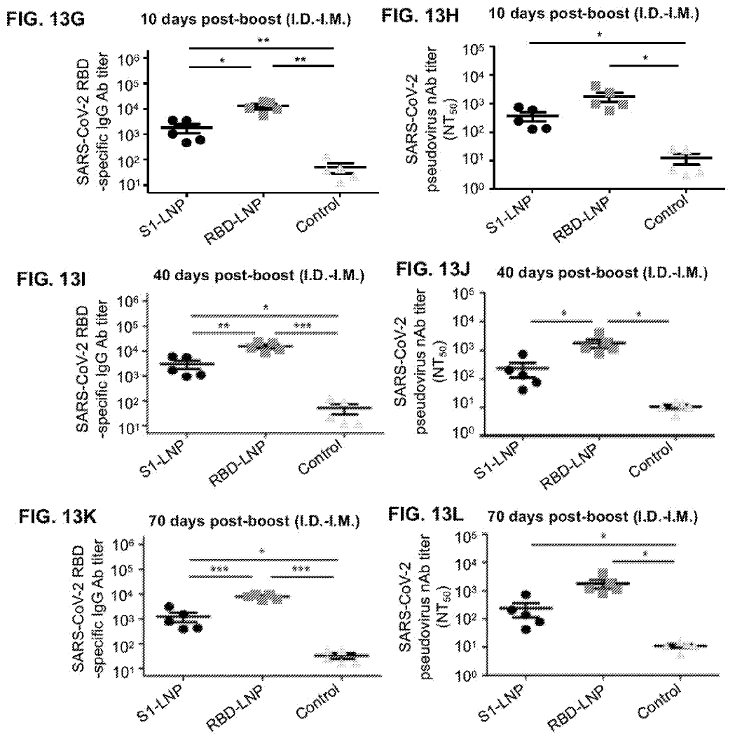

Second, S1 and RBD mRNA-LNPs at 10 μg via I.D. prime and boost induced SARS-CoV-2 RBD-specific IgG (FIG. 13A) and neutralizing antibodies against SARS-CoV-2 pseudovirus (FIG. 13B) 10 days after boost dose, and maintained at similarly high levels for at least 40 and 70 days post-boost immunization (FIG. 13C-F). The neutralizing antibody titer induced by RBD mRNA-LNP was significantly higher than that by 51 mRNA-LNP throughout (FIG. 13A-F).

Third, RBD mRNA-LNPs at 10 μg via I.D. prime and I.M. boost also elicited significantly higher specific IgG and neutralizing antibodies than S1 mRNA-LNPs (FIG. 13G-H) 10 days after boost immunization, and such antibody titers were maintained at similar or even higher levels for at least 70 days post-boost dose (FIG. 13I-L). In contrast, control LNPs only elicited a background, or undetectable, levels of antibody incapable of neutralizing SARS-CoV-2 infection (FIG. 13A-L). Together, these data indicate that RBD mRNA-LNP vaccine immunized at different immunogen doses and different routes of administration could induce strong antibody responses and potent neutralizing antibodies against SARS-CoV-2 infection.

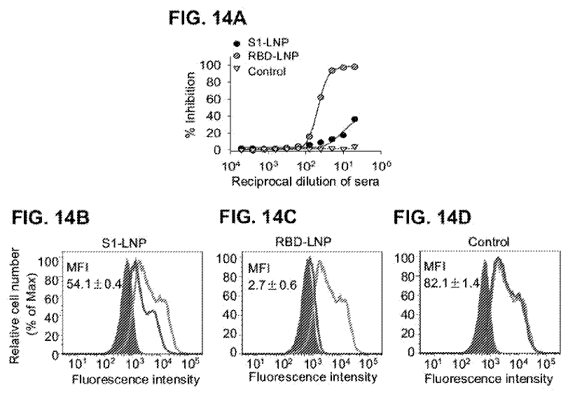

The binding of SARS-CoV-2 RBD to ACE2 receptor in hACE2/293T cells could be inhibited with serum antibodies induced by mRNA-LNP immunization. Specifically, flow cytometry results indicated that 1) anti-RBD antibodies potently inhibited binding between SARS-CoV-2 RBD and its ACE2 receptor (FIG. 14A-B), 2) such inhibition was much stronger than that by similarly induced anti-S1 antibodies (FIG. 14A-C), and 3) such inhibition was dose-dependent (FIG. 14A). In contrast, control LNP-induced mouse sera did not inhibit RBD-ACE2 binding (FIG. 14A, D). These data indicate that SARS-CoV-2 RBD mRNA-LNP-induced antibodies can potently block binding between SARS-CoV-2 RBD and its ACE2 receptor.

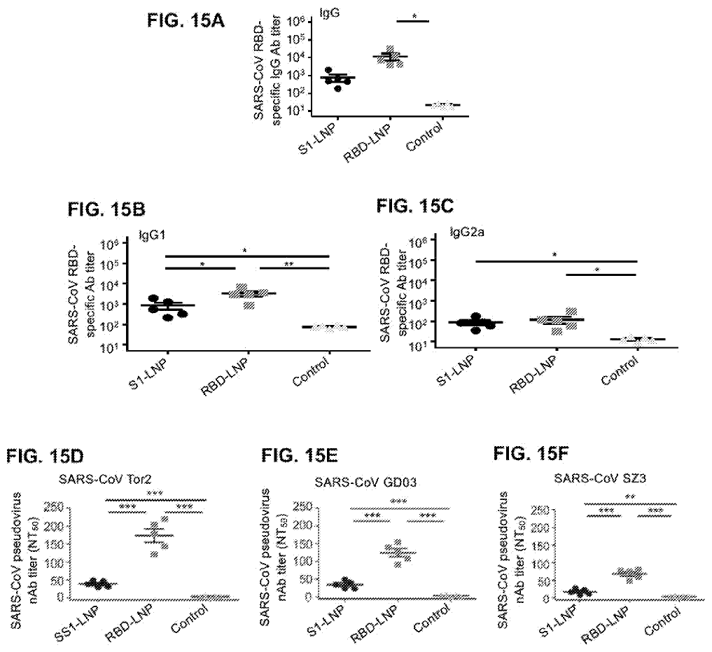

Since SARS-CoV-2 RBD shares about 70% sequence identity with SARS-CoV RBD, we evaluated whether SARS-CoV-2 mRNA-LNP-induced serum antibodies might cross-react with SARS-CoV RBD and neutralize SARS-CoV infection. ELISA results showed that SARS-CoV-2 RBD mRNA-LNP did elicit higher, or significantly higher, titer of IgG (FIG. 15A), IgG1 (FIG. 15B), and IgG2a (FIG. 15C) antibodies compared to SARS-CoV-2 S1 mRNA-LNP in cross-reacting with SARS-CoV RBD. In addition, SARS-CoV-2 RBD mRNA-LNP-induced antibodies had significantly higher titer than those induced by SARS-CoV-2 S1 mRNA-LNP in cross-neutralizing infection of three SARS-CoV pseudoviruses expressing S proteins (with or without RBD variants) of human strains Tor2 (FIG. 15D), GDO3 (FIG. 15E), and palm civet strain SZ3 (FIG. 15F). These data indicate that SARS-CoV-2 RBD mRNA vaccine induced antibodies cross-reacting with and cross-neutralizing SARS-CoV infection.

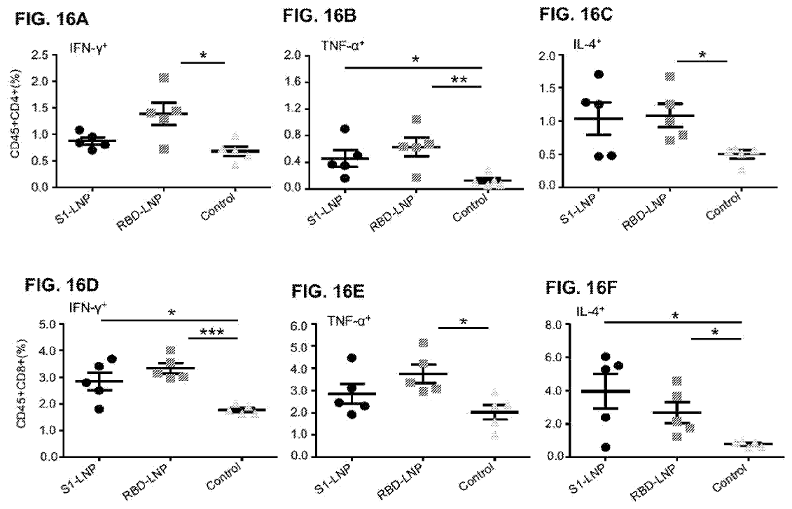

We further investigated SARS-CoV-2 RBD-specific T cell responses induced by LNP-encapsulated SARS-CoV-2 S1 and RBD mRNAs in immunized mice. Splenocytes were collected 10 days post-2nd immunization, stimulated with SARS-CoV-2 RBD overlapping peptides (Table 1), and detected for secretion of IFN-γ (Th1), TNF-α (Th1), and IL-4 (Th2) in CD45-positive (CD45⁺)-CD4⁺ T cells, as well as IFN-γ, TNF-α, and IL-4 in CD45⁺-CD8⁺ T cells by flow cytometric analysis. Compared with the control, RBD mRNA-LNPs could significantly increase the frequency of IFN-γ-, TNF-α- or IL-4-producing CD45⁺-CD4⁺ (FIG. 16A-C) or CD45⁺-CD8⁺ (FIG. 16D-F) T cells, respectively. However, S1 mRNA-LNPs only significantly increased the frequency of TNF-α-producing CD45⁺-CD4⁺ (FIG. 16B) and that of IFN-γ- or IL-4-producing CD45⁺-CD8⁺ (FIG. 16D, 16F) T cells, respectively. Therefore, RBD mRNA vaccine can effectively elicit SARS-CoV-2 RBD-specific CD45⁺-CD4⁺ (Th1) and CD45⁺-CD8⁺ T cell responses.

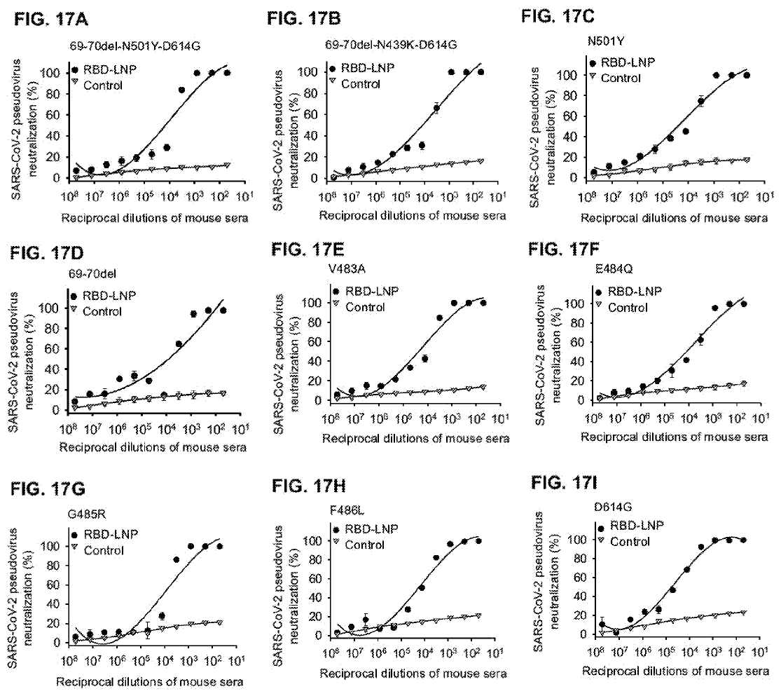

A number of mutations or deletions have been identified in the spike protein, including the RBD, of recently emerged SARS-CoV-2 strains. Several of these mutations or deletions (such as H69-V70del, N501Y, and D614G) are found in the UK, South African, Japan, or Brazil variant strains. About eight months post-2nd immunization, mice immunized with RBD mRNA-LNPs or PBS were injected (I. D.) with the same immunogen, and sera were collected to detect neutralizing activity against infection of pseudotyped SARS-CoV-2 containing single amino acid mutations or combinations of different amino acid mutations in the viral spike protein. The results showed that all of these pseudoviruses tested can be neutralized by the sera of mice immunized with RBD mRNA-LNPs, but not from those of the control mice injected with PBS (FIG. 17A-I). These results demonstrate the ability of SARS-CoV-2 RBD mRNA vaccine in eliciting long-term and broad neutralizing antibodies against multiple SARS-CoV-2 variants.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A severe acute respiratory syndrome (SARS) coronavirus 2 (SARS-CoV-2) immunogenic composition comprising or encoding a polypeptide comprising a receptor binding domain (RBD) domain of a SARS-CoV-2 spike (S) protein.

2. The SARS-CoV-2 immunogenic composition of claim 1, wherein the polypeptide comprising the RBD comprises the S1 domain of the SARS-CoV-2 S protein or an immunogenic fragment thereof.

3. The SARS-CoV-2 immunogenic composition of claim 2, wherein the polypeptide comprising the RBD comprises residues 14-660 of the S1 domain of the SARS-CoV-2 S protein.

4. The SARS-CoV-2 immunogenic composition of claim 3, wherein the amino acid sequence of residues 14-660 of the S1 domain of the SARS-CoV-2 S protein is SEQ ID NO: 4.

5. The SARS-CoV-2 immunogenic composition of any one of claims 1-3, wherein the RBD comprises residues 331-524 of the SARS-CoV-2 S protein.

6. The SARS-CoV-2 immunogenic composition of claim 5, wherein the amino acid sequence of residues 331-524 of the SARS-CoV-2 S protein is SEQ ID NO: 2.

7. The SARS-CoV-2 immunogenic composition of any one of claims 1-6, wherein the immunogenic composition comprises the polypeptide comprising the RBD.

8. The SARS-CoV-2 immunogenic composition of claim 7, wherein the polypeptide comprising the RBD further comprises an IgG Fc domain.

9. The SARS-CoV-2 immunogenic composition of any one of claims 1-8, wherein the immunogenic composition comprises a polynucleotide encoding the polypeptide comprising the RBD.

10. The SARS-CoV-2 immunogenic composition of claim 9, wherein the polynucleotide is a DNA molecule.

11. The SARS-CoV-2 immunogenic composition of claim 9, wherein the polynucleotide is a mRNA molecule.

12. The SARS-CoV-2 immunogenic composition of claim 11, wherein the mRNA comprises pseudouridine in place of uridine.

13. The SARS-CoV-2 immunogenic composition of claim 11 or 12, wherein the mRNA is encapsulated in a lipid nanoparticle.

14. The SARS-CoV-2 immunogenic composition of any one of claims 1-13, further comprising an adjuvant.

15. The SARS-CoV-2 immunogenic composition of any one of claims 1-14 for treating or preventing infection with SARS-CoV-2.

16. A method of treating or preventing infection with SARS-CoV-2 comprising administering to a subject in need thereof the SARS-CoV-2 immunogenic composition according to any one of claims 1-14.

17. The method of claim 16, comprising administering a priming dose and a boosting dose of the immunogenic composition, wherein the boosting dose is administered 3-4 weeks after the priming dose.

18. The method of claim 17, wherein the immunogenic composition is administered intramuscularly, intradermally, intranasally, or subcutaneously.
